(19) United States
(12) Patent Application Publication
(10) Pub. No.: US 2024/0058349 A1
(43) Pub. Date: Feb. 22, 2024

(54) PI3K INHIBITORS FOR PROTECTION AGAINST GLUCOCORTICOID-INDUCED ATROPHY

(71) Applicant: Northwestern University, Evanston, IL (US)

(72) Inventor: Irina Budunova, Chicago, IL (US)

(21) Appl. No.: 18/257,261

(22) PCT Filed: Dec. 16, 2021
(86) PCT No.: PCT/US2021/063832

§ 371 Date: Jun. 13, 2023

Related U.S. Application Data
(60) Provisional application No. 63/127,578, filed on Dec. 18, 2020.

(51) Int. Cl. A61K 31/5377; A61K 31/585; A61K 31/436; A61K 31/573; A61P 19/10; A61P 35/00; A61P 17/00

(52) U.S. Cl. CPC A61K 31/5377; A61K 31/585; A61K 31/436; A61K 31/573; A61P 19/10; A61P 35/00; A61P 17/00

(57) ABSTRACT

Provided herein are methods of treating and preventing steroid-induced atrophy (e.g., skin atrophy, osteoporosis, etc.) by the administration of PI3K/Akt/mTOR inhibitors and pharmaceutical compositions and combinations therefor.

### STATEMENT REGARDING FEDERAL FUNDING

This invention was made with government support under GM112945 and AI125366, awarded by the National Institutes of Health. The government has certain rights in the invention.

### SEQUENCE LISTING

The text of the computer readable sequence listing filed herewith, titled “39041-601_SEQUENCE_LISTING_ST25”, created Dec. 16, 2021, having a file size of 7,473 bytes, is hereby incorporated by reference in its entirety.

### FIELD

Provided herein are methods of treating and preventing steroid-induced atrophy (e.g., skin atrophy, osteoporosis, etc.) by the administration of PI3K/Akt/mTOR inhibitors and pharmaceutical compositions and combinations therefor.

### BACKGROUND

Though the treatment of hematological malignancies has evolved over the past decades, glucocorticoids (Gcs) remain the essential component of combination therapies, and the glucocorticoid receptor (GR) is a well-recognized target for blood cancers (Refs. 1,2; incorporated by reference in their entireties). Unfortunately, patients chronically treated with Gcs develop numerous metabolic and atrophic adverse effects including osteoporosis, muscle waste, skin atrophy that strongly affect their life quality (Refs. 2-4; incorporated by reference in their entireties). Thus, there is a great need for the development of safer GR-targeted therapies.

GR is a transcription factor that regulates gene expression via transactivation (TA) that requires GR binding to the responsive elements in gene promoters, and transrepression (TR) that is mediated, among other mechanisms, by binding and blocking other TFs including pro-inflammatory NF-kB and AP-1 (Refs. 2,5,6; incorporated by reference in their entireties). GR TR is an important mechanism underlying therapeutic anti-inflammatory and anti-cancer effects of Gcs, and it usually does not require GR DNA-binding (Ref. 7; incorporated by reference in its entirety). GR TA regulates GR signaling linked to gluconeogenesis and catabolism, and often mediates steroid atrophic effects in different tissues (Refs. 2,8; incorporated by reference in their entireties). Thus, for years the major efforts in the field have been directed towards the development of selective GR agonists/modulators (SEGRAM) that shift GR activity towards TR (Ref. 9; incorporated by reference in its entirety).

### SUMMARY

Provided herein are methods of treating and preventing steroid-induced atrophy (e.g., skin atrophy, osteoporosis, etc.) by the administration of PI3K/Akt/mTOR inhibitors and pharmaceutical compositions and combinations therefor.

In some embodiments, provided herein are methods of treating, preventing, and/or reducing glucocorticoid-induced side-effects in a subject comprising administering to the subject a PI3K/Akt/mTOR inhibitor. In some embodiments, the PI3K/Akt/mTOR inhibitor is co-administered with the glucocorticoid. In some embodiments, the PI3K/Akt/mTOR inhibitor is administered to a subject that has previously been administered the glucocorticoid. In some embodiments, the subject exhibits glucocorticoid-induced side-effects. In some embodiments, the subject has not yet exhibited glucocorticoid-induced side-effects. In some embodiments, the glucocorticoid-induced side-effects are selected from osteoporosis, skin atrophy, muscle atrophy, metabolic disorder, and endocrine disorder. In some embodiments, the PI3K/Akt/mTOR inhibitor is selected from sirolimus, wortmannin, alvocidib, AZD8055, triptolide, WYE-125132, OSI-027, CGP-60474, PI-103, BMS-387032, emetine, NVP-BEZ235, torin 1, LY294002, QL-X-138, and mitoxantrone. In some embodiments, the PI3K/Akt/mTOR inhibitor is selected from rapamycin, OSI-027, wortmannin, LY294002, NVP-BEZ235, AZD8055, and MK-2206. In some embodiments, the PI3K/Akt/mTOR inhibitor is selected from:

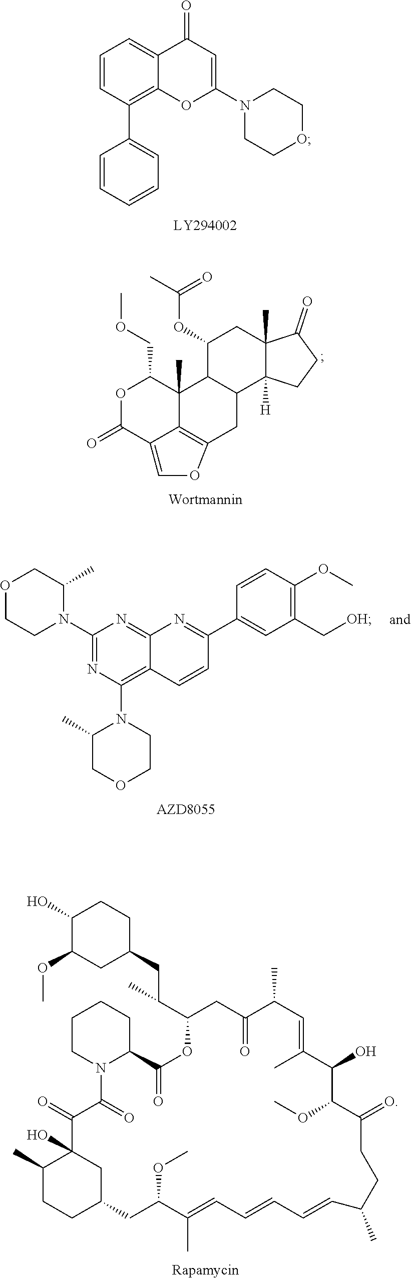

In some embodiments, the PI3K/Akt/mTOR inhibitor is a PI3K inhibitor, an Akt inhibitor, or an mTOR inhibitor.

In some embodiments, provided herein are kits or pharmaceutical compositions comprising an PI3K/Akt/mTOR inhibitor and a glucocorticoid agent.

In some embodiments, provided herein are compositions comprising a PI3K/Akt/mTOR inhibitor for use in a method of ng, preventing, and/or reducing glucocorticoid-induced side-effects in a subject. In some embodiments, the PI3K/Akt/mTOR inhibitor is co-administered with the glucocorticoid. In some embodiments, the PI3K/Akt/mTOR inhibitor is administered to a subject that has previously been administered the glucocorticoid. In some embodiments, the subject exhibits glucocorticoid-induced side-effects. In some embodiments, the subject has not yet exhibited glucocorticoid-induced side-effects. In some embodiments, the glucocorticoid-induced side-effects are selected from osteoporosis, skin atrophy, muscle atrophy, metabolic disorder, and endocrine disorder. In some embodiments, the PI3K/Akt/mTOR inhibitor is selected from sirolimus, wortmannin, alvocidib, AZD8055, triptolide, WYE-125132, OSI-027, CGP-60474, PI-103, BMS-387032, emetine, NVP-BEZ235, torin 1, LY294002, QL-X-138, and mitoxantrone. In some embodiments, the PI3K/Akt/mTOR inhibitor is selected from rapamycin, OSI-027, wortmannin, LY294002, NVP-BEZ235, AZD8055, and MK-2206. In some embodiments, the PI3K/Akt/mTOR inhibitor is selected from:

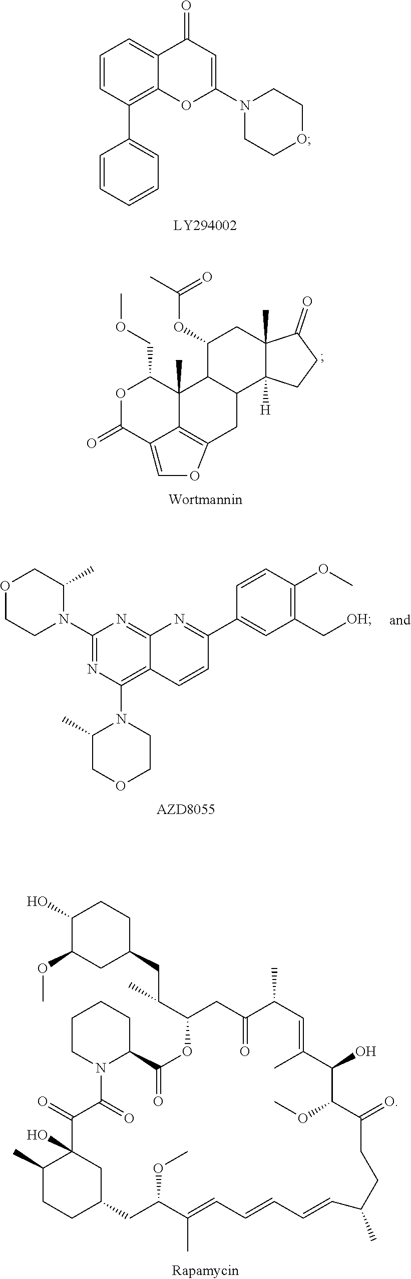

In some embodiments, the PI3K/Akt/mTOR inhibitor is a PI3K inhibitor, an Akt inhibitor, or an mTOR inhibitor.

### BRIEF DESCRIPTION OF THE DRAWINGS

FIG. 1A-D. PI3K/mTOR/Akt inhibitors suppress the expression of REDD1 in CEM and Granta cells. CEM (A-B) and Granta cells (C-D) were pretreated with solvent (Control), LY294002 (50 uM), AZD8055 (1 uM) and WM (10 uM) for 6 h and treated with either solvent or glucocorticoid Dex (1 uM) for 24 h. (A, C) Q-PCR analysis of REDD1 mRNA expression in CEM (A) and Granta (C) cells. The Q-PCR results were normalized to the expression of housekeeping gene RPL27, and presented as fold change compared to control. The mean+/−SD was calculated for three individual RNA samples/condition. Statistically significant differences as compared to: a-control; b-Dex (p<0.001) as and where indicated. (B, D) Western blot analysis of REDD1 protein levels, phosphorylation of mTOR downstream substrate 4-EBP1 and Akt phosphorylation at Thr308 in CEM (B) and Granta (D) cells. GAPDH was used as the loading controls.

FIG. 2A-I. PI3K/mTOR/Akt inhibitors shift GR function towards transrepression. CEM (A, E) and Granta (C, G) cells stably expressing GRE. Luciferase and NF-κB. Luciferase were pretreated with LY294002 (50 uM), AZD8055 (1 uM) and WM (10 uM) for 6 h and treated with either solvent or glucocorticoid Dex (1 uM) for 24 h. (A, C, E, G) Luciferase assay. Luciferase activity is presented as mean+/−SD for three individual wells/point. (B, D, F, H) Q-PCR analysis of the expression of GR- and NF-kB regulated endogenous genes. Q-PCR results for repressed genes (CCND1, CCND2, IL7R, CD86) and activated genes (FKBP51, GILZ) were normalized to the expression of housekeeping gene RPL27, and presented as fold change compared to control. Cells were treated as above. The mean+/−SD was calculated for three individual RNA samples/condition. Statistically significant differences as compared to: a-control; b-Dex (p<0.001) as and where indicated. (I) Western blot analysis of GR phosphorylation. Cells were treated as above. GAPDH was used as loading control.

FIG. 3A-J. Synergistic and additive cytotoxic effects of PI3K/mTOR/Akt inhibitors and Dexamethasone in CEM and Granta cells. (A-E) Cytotoxic effect was determined using MTT assay. CEM (A) and Granta (E) cells were pretreated with solvent (Control), LY294002 (50 uM), AZD8055 (1 uM) and WM (10 uM) for 6 h and treated with either solvent or glucocorticoid Dex (1 uM) for 24-144 h. (B-D) Isobologram analysis in CEM cells for the combination of LY294002 (B), AZD8055 (C), WM (D) and Dex. (F-H) Isobologram analysis in Granta cells for the combination of LY294002 (F), AZD8055 (G), WM (H) and Dex. The concentration, which resulted in 50% cell growth inhibition (IC50), is expressed as 1.0 on X and Y axis of the isobologram. Y-axis: LY294002, AZD8055 or WM (IC50); X-axis: Dex (IC50). (J, K) Apoptosis induction was evaluated by flow cytometry using PI staining (J) and by Western blot analysis of cleaved PARP level (K). GAPDH was used as loading control. The mean+/−SD was calculated for three individual samples/condition. Statistically significant differences as compared to: a-control; b-Dex (p<0.001) as and where indicated.

FIG. 4A-G. Cooperative anti-cancer effects of PI3K/mTOR/Akt inhibitors and Dexamethasone in lymphoma xenograft model. Subcutaneous xenografts were established using Granta cells. Mice (10 animals/group) were treated every 48 h with i.p. injections of LY294002 (20 mg/kg), Rapa (5 mg/kg) or vehicle (30% PEG3350, 4% DMSO, 5% Tween-20 in PBS) followed by i.p. injections of Dex (1 mg/kg) of vehicle (5% DMSO in PBS) 6 h later. Control mice were monitored until the tumor size reached 1500 mm³ at which point they were euthanized. (A) Tumor volume on 27^{th} day of treatment. (B) H&E staining. (C) Quantification of cellularity in xenografts. (D, F) Immunohistochemical staining of xenograft samples for Ki67 (D), and caspase 3 (F). Scale bars: 40 um. (E, G) Quantification of Ki67—(E) and caspase 3—(G) positive cells. At least five individual fields per slide of each individual sample/treatment (at least 50 images/treatment group) were analyzed using Axioplan2 microscope software (Carl Zeiss). The cell density of xenografts and the number of Ki67- and caspase 3-positive cells in treated animals is presented as % to control. In A, C, E and G bar graphs the mean+/−SD is presented. Statistically significant differences as compared to: a-control; b-Dex (p<0.001) as and where indicated.

FIG. 5A-E. Protective effect of LY294002 and Rapamycin against atrophic effects of systemic chronic Dex treatment in bone and skin. 12 weeks old female BALB/c mice (10/group) were treated every 48 h with i.p. injections of LY294002 (20 mg/kg) or vehicle (30% PEG3350, 4% DMSO, 5% Tween-20 in PBS) followed by i.p. injections of Dex (10 mg/kg) of vehicle (5% DMSO in PBS) 6 h later for 5 weeks. (A) Quantification of cortical bone thickness. (B) Q-PCR analysis of Rankl and Opg mRNA expression in bone tissue. The Q-PCR results (for three individual RNA samples/condition) were normalized to the expression of housekeeping gene RPL27, and presented as fold change compared to control. (C) H&E skin staining. (D, E) Quantification of the epidermal (D) and dermal adipose (E) thickness as described in Materials and Methods. In bar graphs the mean+/−SD is presented. Statistically significant differences as compared to: a-control; b-Dex (p<0.001) as and where indicated.

FIG. 6. Quantification of Western blot analysis. CEM cells (A,C,E) and Granta cells (B,D,F) were pretreated with solvent (Control), LY294002 (50 uM), AZD8055 (1 uM) and WM (10 uM) for 6 h and treated with either solvent or glucocorticoid Dex (1 uM) for 24 h. Quantification of Western blot analysis of REDD1, p-4EBP1, p-Akt(A,B), p-GR (C,D) and cleaved PARP (E,F) protein levels was done using Image J free software. Statistically significant differences as compared to: a-control; b-Dex(p<0.001) as and where reflected.

FIG. 7. Effect of PI3K/mTOR/Akt inhibitors on regulation of endogenous genes by Dex in CEM and Granta cells. CEM (A) and Granta (B) cells were pretreated with solvent (Control), LY294002 (50 uM), AZD8055 (1 uM) and WM (10 uM) for 6 h and treated with either solvent or glucocorticoid Dex (1 uM) for 24 h. Q-PCR results for TA-associated genes (KLF9,MKP1) were normalized to the expression of house keeping gene RPL27. (C,D) Western blotting (C) of GR nuclear translocation and its densitomeric analysis (D). CEM and Granta cells were pretreated with solvent (Control), LY294002 (50 uM) or AZD8055 (luM) for 6 h and treated with either solvent or glucocorticoid Dex (luM) for 24 h. Histone H3 was used as loading control. Experiments were done in triplicates. Statistically significant differences as compared to: a-control; b-Dex (p<0.001) as and where reflected.

FIG. 8. Cytotoxic effects of LY294002 (A), Wortmannin (B) and AZD8055 (C) on CEM and Granta cells. CEM and Granta cells were pretreated with solvent (Control), LY294002 (1-500 uM), WM (0.1-25 uM) and AZD8055 (0.05-5 uM) for 6 h and treated with either solvent or glucocorticoid Dex (1 uM) for 24 h. MTT assay was performed in triplets. IC50 values were calculated using QuestGraph IC50 Calculator free software.

FIG. 9. Comparative cytotoxic effects of LY294002 (A), Wortmannin (B) and AZD8055 (C) on CEM cells, Granta cells and normal human monocytes. CEM cells, Granta cells and normal human monocytes were pretreated with solvent (Control), LY294002 (50 uM), WM (10 uM) and AZD8055 (1 uM) for 6 h and treated with either solvent or glucocorticoid Dex (1 uM) for 24 h. MTT assay was performed in triplets. Statistically significant differences as compared to: c-CEM cells; d-Granta cells (p<0.001) as and where reflected.

FIG. 10. Synergistic anti-lymphoma effect of Rapamycin and Dex in CEM and Granta cells. (A,D) Cytotoxic effect was determined using MTT assay. CEM (A) and Granta (D) cells were pretreated with solvent (Control), Rapa (1 uM) for 6 h and treated with either solvent or glucocorticoid Dex (1 uM) for 24-144 h. (B,E) Isobologram analysis in CEM (B) and Granta (E) cells for the combination of Rapa and Dex. The concentration, which produced 50% cell growth inhibition (IC50), is expressed as 1.0 in the ordinate and the abscissa of the isobologram. Y-axis, LY294002, AZD8055 or WM(IC50); X-axis, Dex (IC50). (C,F) Apoptosis induction was evaluated by flow cytometry using PI staining. Statistically significant differences as compared to: a-control; b-Dex (p<0.001) as and where reflected.

FIG. 11. Effect of LY294002, Rapamycin and Dex on animal body weight in xenograft study. Granta cells were subcutaneously injected into the right flank of 7 weeks old female nu/nu mice. Animals were randomized and treated i.p. every 48 h with LY294002 (20 mg/kg), Rapamycin (5 mg/kg) or vehicle (30% PEG3350, 4% DMSO, 5% Tween 20 in PBS) followed by Dex (1 mg/kg) of vehicle (5% DMSO in PBS) 6 h later. Body weight was recorded twice a week. Body weight was measured twice a week. There were no statistically significant differences in body weight changes between experimental and control groups.

FIG. 12. Anti-tumor effect of Dex, Rapa, LY294002 on Granta xenografts. Granta cells were subcutaneously injected into the right flank of 7 weeks old female nu/nu mice. Animals were randomized and treated i.p. every 48 h with LY294002 (20 mg/kg), Rapamycin (5 mg/kg) or vehicle (30% PEG3350, 4% DMSO, 5% Tween 20 in PBS) followed by Dex (1 mg/kg) of vehicle (5% DMSO in PBS) 6 h later. Tumor size was measured twice a week by digital calipers. Treatment groups receiving Rapa or LY294002 individually exhibited statistically significant tumor growth inhibition, relative to the control group, from day 12; treatment groups receiving Rapa or LY294002 in combination with Dex exhibited statistically significant tumor growth inhibition, relative to the control group (p<0.05), from day 12, relative to Dex-treated group, from day 21 (p<0.05).

FIG. 13. Effect of LY294002, Rapamycin and Dex on animal body weight in Dexamethasone-induced osteoporosis study. GIOP was induced by i.p. injection of Dex (10 mg/kg) in 12 weeks old BALB/c female mice every 48 h for 5 weeks. 6 h prior to Dex, animals were treated i.p. with LY294002 (20 mg/kg), Rapamycin (5 mg/kg) of vehicle (30% PEG3350, 4% DMSO, 5% Tween-20 in PBS). Body weight was measured twice a week

FIG. 14. Q-PCR analysis of Col1a1 and Col2a1 mRNA expression in bone tissue. GIOP was induced by i.p. injection of Dex (10 mg/kg) in 12 weeks old BALB/c female mice every 48 h for 5 weeks. 6 h prior to Dex, animals were treated i.p. with LY294002 (20 mg/kg), Rapamycin (5 mg/kg) of vehicle (30% PEG3350, 4% DMSO, 5% Tween 20 in PBS). Animals were euthanized 24 h after the last Dex treatment. Total RNA from bone tissue was isolated with RiboPure RNA purification kit and cleaned with TURBODNase. Col1a1 and Col2a1 primers were designed with NCBI Primer-BLAST (Table 1). Results were normalized to the expression of the housekeeping RPL27 gene. Statistically significant differences as compared to: a-control; b-Dex (p<0.001) as and where reflected.

FIG. 15. Rapamycin prevents induction of atrophogene REDD1 by Dexamethasone in murine osteocytes

### DEFINITIONS

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In some embodiments, the subject may be over the age of 50.

As used herein, the terms “treat,” “treatment,” and “treating” refer to reducing the amount or severity of a particular condition, disease state (e.g., steroid-induced atrophy), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular symptom, condition, or disease state from occurring in a subject not presently experiencing or afflicted with the condition or disease state. The terms do not necessarily indicate complete or absolute prevention. For example, “prevention” refers to reducing the likelihood of a symptom, condition, or disease state from occurring in a subject not presently experiencing or diagnosed with the symptom, condition, or disease state. In order to “prevent” a symptom, condition, or disease state, a composition or method need only reduce the likelihood of the symptom, condition, or disease state, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided herein are methods of treating and preventing steroid-induced atrophy (e.g., skin atrophy, osteoporosis, etc.) by the administration of PI3K/Akt/mTOR inhibitors and pharmaceutical compositions and combinations therefor.

Glucocorticoids are widely used for therapy of hematological malignancies. Unfortunately, chronic treatment with glucocorticoids commonly leads to adverse effects including skin and muscle atrophy and osteoporosis. Experiments conducted during development of embodiments herein demonstrated that REDD1 (regulated in development and DNA damage 1) plays central role in steroid atrophy. Experiments were conducted during development of embodiments herein to determine whether REDD1 suppression makes glucocorticoid-based therapy of blood cancer safer. ˜50% of top putative REDD1 inhibitors selected by bioinformatics screening of LINCS database were PI3K/Akt/mTOR inhibitors. Wortmannin, LY294002 and AZD8055 were selected as exemplary PI3K/Akt/mTOR inhibitors, and experiments demonstrated that they blocked basal and glucocorticoid-induced REDD1 expression. Moreover, all PI3K/mTOR/Akt inhibitors tested modified glucocorticoid receptor function, shifting it towards therapeutically important transrepression. PI3K/Akt/mTOR inhibitors enhanced anti-lymphoma effects of Dexamethasone in vitro and in vivo, in lymphoma xenograft model. The therapeutic effects of PI3K inhibitor+Dexamethasone combinations ranged from cooperative to synergistic, especially in case of LY294002 and Rapamycin, used as a previously characterized reference REDD1 inhibitor. Co-administration of LY294002 or Rapamycin with Dexamethasone protected skin against Dexamethasone-induced atrophy, and normalized RANKL/OPG ratio indicating a reduction of Dexamethasone-induced osteoporosis. These results demonstrate a safer and more effective glucocorticoid-based combination therapy of hematological malignancies using PI3K/Akt/mTOR inhibitors.

Recently mTOR/Akt inhibitor REDD1 (Regulated in development and DNA damage 1) was identified as the key mediator of Gcs-induced atrophy in different tissues. Indeed, in REDD1 KO animals muscle and skin were protected against Gcs atrophy (Refs. 10,11; incorporated by reference in their entireties). In REDD1 KO mice, the anti-inflammatory potential of Gcs was completely preserved (Ref. 10; incorporated by reference in its entirety), suggesting that inhibitors of REDD1 expression could dissociate therapeutic and adverse atrophogenic effects of Gcs.

mTOR/Akt inhibitor REDD1 (Regulated in development and DNA damage 1) was identified as the key mediator of Gcs-induced atrophy in different tissues. Indeed, in REDD1 KO animals muscle and skin were protected against Gcs atrophy (Refs. 10,11; incorporated by reference in their entireties). In REDD1 KO mice, the anti-inflammatory potential of Gcs was completely preserved (Ref. 10; incorporated by reference in its entirety), suggesting that inhibitors of REDD1 expression could dissociate therapeutic and adverse atrophogenic effects of Gcs.

Prior to the present work, no REDD1 pharmacological inhibitors were known. A drug repurposing approach was utilized using bioinformatics screening of Library of Integrated Network-Based Cellular Signatures (LINCS) database of ˜20,000 transcriptional signatures induced by FDA-approved and experimental drugs (lincsproject.org/LINCS/). A significant number of putative inhibitors of REDD1 expression were identified among PI3K/Akt/mTOR inhibitors (Ref 12,13; incorporated by reference in their entireties) including classical experimental inhibitors such as LY294002 and Wortmannin (WM), and drugs in clinical trials such as Rapamycin (Rapa) and AZD8055. This was completely unexpected as PI3K/Akt/mTOR inhibitors block major pro-proliferative signaling in cells. Experiments conducted during development of embodiments herein demonstrated that compounds from this pharmacological class inhibited REDD1 in keratinocytes and attenuated skin atrophy induced by topical Gcs but did not interfere with their anti-inflammatory effects (Refs. 12,13; incorporated by reference in their entireties).

Experiments were conducted during development of embodiments herein to evaluate whether the combination of Gcs and PI3K/Akt/mTOR inhibitor is applicable to increase safety of systemic Gcs used for blood cancer. The effects of PI3K/Akt/mTOR inhibitors AZD8055, WM and LY294002 on REDD1 expression and GR function were analyzed in leukemia and lymphoma cells. Atrophogenic effects of systemic Dexamethasone (Dex)+/−LY294002 or Rapa, previously validated as REDD1 inhibitor (Ref. 13; incorporated by reference in its entirety), were assessed using osteoporosis and skin atrophy mouse models. The anti-lymphoma effects of this combination therapy were also monitored in human lymphoma xenograft model to assure that the regimens chosen for tissues protection remain optimal for anti-cancer activity. It was demonstrated that the exemplary combination of LY294002 and Rapa prevented development of Dex-induced osteoporosis and skin atrophy, and enhanced Dex anti-lymphoma effects.

In some embodiments, provided herein are methods of reducing side effects (e.g., atrophy (e.g., osteoporosis)) associated with glucocorticoid administration and pharmaceutical compositions and combinations (e.g., glucocorticoids and PI3K/Akt/mTOR inhibitors) therefore. In some embodiments, a subject is administered a PI3K/Akt/mTOR inhibitor. In some embodiments, the PI3K/Akt/mTOR inhibitor is selected from sirolimus, wortmannin, alvocidib, AZD8055, triptolide, WYE-125132, OSI-027, CGP-60474, PI-103, BMS-387032, emetine, NVP-BEZ235, torin 1, LY294002, QL-X-138, and mitoxantrone. In some embodiments, the PI3K/Akt/mTOR inhibitor is selected from rapamycin, OSI-027, wortmannin, LY294002, NVP-BEZ235, AZD8055, and MK-2206. In some embodiments, a PI3K/Akt/mTOR inhibitor is selected from

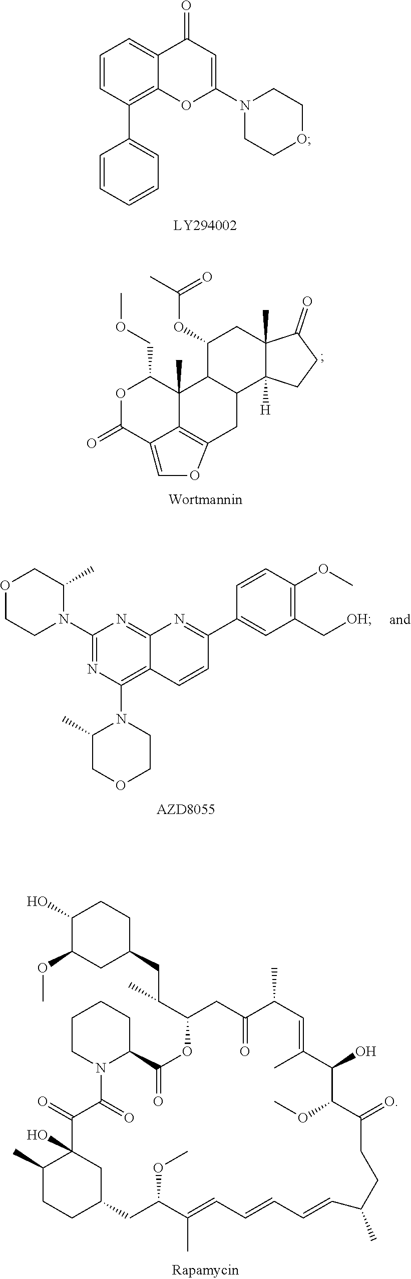

In some embodiments, administration or formulations comprising a PI3K/Akt/mTOR inhibitor comprise an mTOR inhibitor, a PI3K inhibitor, and/or a Akt1 inhibitor.

In some embodiments, an mTOR inhibitor is administered to a subject (e.g., a subject that has received or is receiving treatment with a glucocorticoid agent). Suitable mTOR inhibitors are selected from rapamycin, temsirolimus, everolimus, and sirolimus.

In some embodiments, a PI3K inhibitor is administered to a subject (e.g., a subject that has received or is receiving treatment with a glucocorticoid agent). Suitable PI3K inhibitors are selected from Wortmannin, LY294002, hibiscone C, idelalisib, copanlisib, duvelisib, alpelisib, taselisib, perifosine, buparlisib, duvelisib, umbralisib, dactolisib, CUDC-907, voxtalisib, ME-401, IPI-549, SF1126, RP6530, INK1117, pictilisib, XL147, palomid 529, GSK1059615, ZSTK474, PWT33597, IC87114, TG100-115, CAL263, RP6503, PI-103, GNE-477, and AEZS-136.

In some embodiments, a Akt1 inhibitor is administered to a subject (e.g., a subject that has received or is receiving treatment with a glucocorticoid agent). Suitable Akt1 inhibitors are selected from A-674563, GSK690693, AZD5363, ipatasertib (GDC-0068), MK-2206 2HCl, AT7867, AT13148, perifosine, AFURESERTIB (GSK2110183), GSK2141795, ARQ 092, ARQ 751, and MK-2206.

In some embodiments, provided herein is the administration of one or more PI3K/Akt/mTOR inhibitors to a subject that has also been administered one or more glucocorticoid agents. In some embodiments, a PI3K/Akt/mTOR inhibitor and a glucocorticoid agent are co-administered.

Glucocorticoids are a type of corticosteroid hormone that is very effective at reducing inflammation and suppressing the immune system. Glucocorticoids are commonly used to treat conditions that have inflammation as a symptom, such as: allergies, arthritis, asthma, autoimmune disorders (e.g., multiple sclerosis, rheumatoid arthritis, etc.), cancer (e.g., hematological malignancies, breast cancer, etc.), inflammatory bowel disease, lichen planus, lupus, postoperative or chemotherapy-induced nausea and vomiting, postoperative swelling or inflammation, psoriasis, severe mouth ulcers, skin inflammation and rashes, ulcerative colitis, etc. In some embodiments, a subject is administered (or has been administered) a glucocorticoid systemically. In some embodiments, a subject is administered (or has been administered) a glucocorticoid over an extended time period (e.g., multiple days (e.g., 3, 4, 5, 6, 7, 8, 9, 10, or more), multiple weeks (e.g., 2, 3, 4, 5, 6, or more), multiple months (e.g., 2, 3, 4, 5, 6, 9 12, or more). In some embodiments, a subject is administered (or has been administered) a glucocorticoid, such as betamethasone, budesonide, dexamethasone, hydrocortisone, methylprednisolone, prednisone, prednisolone, triamcinolone, etc.

In some embodiments, a subject administered a glucocorticoid suffers from or is at risk of steroid-induced side effects (e.g., steroid-induced atrophy). Glucocorticoids are commonly used to treat a variety of diseases and conditions. Glucocorticoid therapy, particularly prolonged and/or high-dose glucocorticoid therapy, has many potential side effects.

Osteoporosis is one of the most common and debilitating side effects associated with glucocorticoid therapy, particularly prolonged and/or high-dose glucocorticoid therapy. Glucocorticoids reduce bone formation and increase bone resorption. In some embodiments, a subject that has been administered a glucocorticoid is administered a PI3K/Akt/mTOR inhibitor to treat or prevent steroid-induced osteoporosis.

Various metabolic and endocrine effects, such as hyperlipidemia and hyperglycemia, are associated with glucocorticoid therapy. In some embodiments, a subject that has been administered a glucocorticoid is administered a PI3K/Akt/mTOR inhibitor to treat or prevent the metabolic and/or endocrine effects of glucocorticoid administration.

Skin, muscle, and/or soft tissue atrophy is an associated side effect of glucocorticoid therapy. In some embodiments, a subject that has been administered a glucocorticoid is administered a PI3K/Akt/mTOR inhibitor to treat or prevent glucocorticoid-associated skin, muscle, and/or soft tissue atrophy.

In some embodiments, a subject is (or has been) administered a glucocorticoid as part of a cancer treatment. A cancer or tumor may arise in any organ or tissue. For example, the cancer or tumor may be a carcinoma (cancer arising from epithelial cells), a sarcoma (cancer arising from bone and soft tissues), a lymphoma (cancer arising from lymphocytes), a melanoma, or brain and spinal cord tumors. The tumor or cancer cells can arise in the oral cavity (e.g., the tongue and tissues of the mouth) and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid). More particularly, tumors or cancers of the digestive system can arise in the esophagus, stomach, small intestine, colon, rectum, anus, liver, gall bladder, and pancreas. Cancers or tumors of the respiratory system can arise in the larynx, lung, and bronchus and include, for example, non-small cell lung carcinoma. Cancers or tumors of the reproductive system can affect the uterine cervix, uterine corpus, ovaries, vulva, vagina, prostate, testis, and penis. Cancers of the urinary system can arise in the urinary bladder, kidney, renal pelvis, and ureter. Cancer cells also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, etc.). The cancer may be a primary tumor, or alternatively, or a metastatic tumor. In some embodiments, the cancer is a head and neck cancer, such as a squamous cell head and neck carcinoma (HNSCC) or unresectable recurrent or metastatic head and neck squamous cell carcinoma (R/M HNSCC). Methods herein may find use in the treatment of (or treatment/prevention of side effects arising from the treatment of) any of the aforementioned cancers, particularly when a glucocorticoid is administered in the treatment regimen.

In some embodiments, provided herein are pharmaceutical compositions and combinations of pharmaceuticals (e.g., kits) intended for and/or suitable for carrying out the methods of treatment/prevention described herein. In some aspects, provided herein are compositions for treating or preventing steroid-induced side effects (e.g., atrophy, osteoporosis, metabolic disorders, etc.) in a subject. In some embodiments, the steroid-induced side effects are associated with administration of glucocorticoids. In some embodiments, pharmaceutical compositions comprise one or more PI3K/Akt/mTOR inhibitors. In some embodiments, compositions comprise one or more PI3K/Akt/mTOR inhibitors and one or more glucocorticoids (e.g., in a pharmaceutical composition, in a kit, etc.). Suitable PI3K/Akt/mTOR inhibitors are described herein.

The PI3K/Akt/mTOR inhibitors may be provided to the subject by any suitable route, including orally or parenterally. In some embodiments, PI3K/Akt/mTOR inhibitors are formulated for any suitable route of administration. The PI3K/Akt/mTOR inhibitors may be provided to the subject at any suitable time point, for any suitable duration, to achieve the desired result. In some embodiments, PI3K/Akt/mTOR inhibitors and glucocorticoids are co-administered together (e.g., co-formulated or separately formulated). In some embodiments, PI3K/Akt/mTOR inhibitors and glucocorticoids are administered at separate time points. In some embodiments, PI3K/Akt/mTOR inhibitors and glucocorticoids are formulated together into a single composition (e.g. pill, topically-administered liquid, inhalant, liquid for parenteral administration, etc.). In some embodiments, the PI3K/Akt/mTOR inhibitors and glucocorticoids formulated together within a composition are configured for separate therapeutic release regimens (e.g. timed release, delayed release, immediate release, etc.). In some embodiments, PI3K/Akt/mTOR inhibitors and glucocorticoids formulated together within a composition are configured for immediate effectiveness. In some embodiments, the PI3K/Akt/mTOR inhibitors and glucocorticoids are formulated as separate compositions to be co-administered. In some embodiments, co-administration comprises administering separate compositions simultaneously, or near simultaneously. In some embodiments, co-administration comprises a therapeutic strategy in which a subject is administered separate compositions, but not necessarily together.

The one or more PI3K/Akt/mTOR inhibitors and glucocorticoids may be provided to the subject at any suitable dose. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual agents, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating clinician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the composition is administered in maintenance doses, ranging from 0.01 μg to 100 g per kg of body weight.

### EXPERIMENTAL

Provided herein is an approach to safer GR-targeted therapies via combination of glucocorticoids with tissue protectors which were selected by the bioinformatics screening of small molecule drugs based on their negative effect on atrophogene REDD1.

### Example 1

Anti-atrophogenic compounds from the pharmacological class of PI3K/Akt/mTOR inhibitors were tested and it was found that PI3K/Akt/mTOR inhibitors potently inhibit the expression of Akt/mTOR genetic regulator REDD1, confirming the existence of a feedback loop in Akt/mTOR signaling. This loop tightly regulates REDD1 expression at RNA/protein levels. mTOR also regulates REDD1 proteasomal degradation and thus, mTOR inhibitors reduce REDD1 protein stability (Ref. 43; incorporated by reference in its entirety).

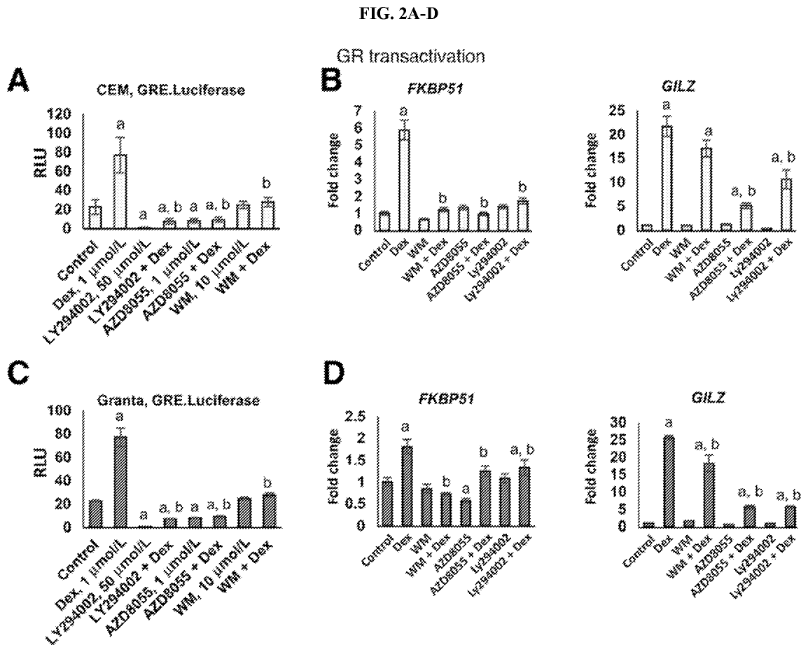

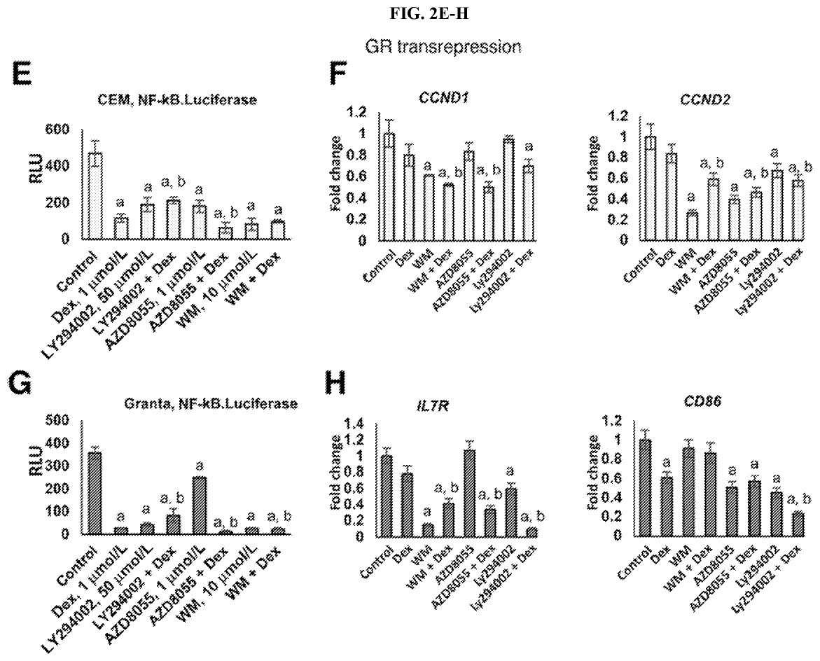

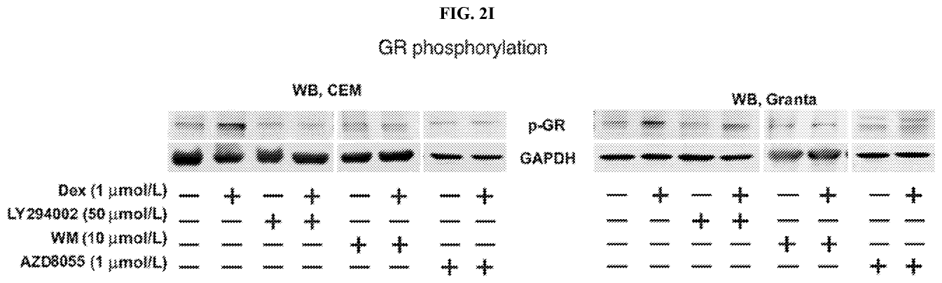

Numerous PI3K/Akt/mTOR inhibitors were shown to modify GR function, shifting overall GR activity towards therapeutically important TR both in lymphoid cell types (FIG. 2), and also in epithelial cells as assessed by Luciferase test and by global changes in Gcs transcriptome (Refs. 12,13; incorporated by reference in their entireties).

The inhibition of GR TA followed inhibition of GR phosphorylation and decreased GR nuclear import. GR is phosphorylated on multiple residues by mitogen-activated protein kinases (MAPKs), cyclin-dependent kinases (CDKs), casein kinase II (CK2) and glycogen synthase kinase-3β (GSK-3β) (Refs. 27,44; incorporated by reference in their entireties). Some PI3K inhibitors such as LY294002 have off-target effects mediated by binding to BET proteins (Bromodomain and Extra-Terminal motif proteins) regulating the histone acetylation. As GR is also involved into epigenetic regulation of gene expression, possible cross-talk between Gcs/GR and BET signaling could affect the expression of GR target genes, especially cytokines and chemokines implicated into anti-inflammatory Gcs response (Refs. 45-47; incorporated by reference in their entireties).

Akt itself can be directly involved in GR phosphorylation and GR function tuning (Ref. 48; incorporated by reference in its entirety). These literature data suggest that PI3K/Akt/mTOR inhibitors affected GR phosphorylation status via GR kinases as mTOR and Akt crosstalk with signaling pathways involving p38, GSK-3beta is known (Refs. 27,44; incorporated by reference in their entireties). At the same time, the exaggeration of GR TR branch (associated with anti-inflammatory and anti-cancer effects of Gcs) in part reflects profound negative effect of PI3K inhibitors on NF-kB activation (Refs. 12,13; incorporated by reference in their entireties).

The directional changes in GR function (down-regulation of GR TA) and blockage of atrophogene REDD1 suggested that PI3K/Akt/mTOR indeed could protect tissues against steroid atrophy. Using two different models of steroid atrophy—skin atrophy and osteoporosis, the protective effects of PI3K inhibitors, when they and Gcs were delivered systemically, were validated.

The PI3K/AKT/mTOR pathway plays a central role in regulating growth and survival of lymphoid cells, and constitutive PI3K activation is frequently observed in different types of lymphoid malignancies (Refs. 49,50; incorporated by reference in their entireties). Combination of Gcs with PI3K inhibitors enhanced anti-lymphoma effects of Dex in vitro and in vivo, in human lymphoma xenograft model, and the therapeutic effects of PI3K inhibitor+Dex combinations ranged from cooperative to synergistic (particularly in case of Rapamycin and LY294002).

Experiments conducted during development of embodiments herein demonstrate the utility of using compounds from PI3K/Akt/mTOR inhibitors class to prevent atrophic effects induced by chronic systemic treatment with Gcs. This combination provided enhanced anti-lymphoma activity of Gcs. In addition to atrophy induced in different tissues, chronic treatment with Gcs results in metabolic, gastrointestinal and cardiovascular side effects (Ref. 53; incorporated by reference in its entirety). As some of those adverse effects including glucose metabolism also strongly depend on TA branch of GR signaling (Ref. 54; incorporated by reference in its entirety), PI3K/Akt/mTOR inhibitors also find use in ameliorating metabolic adverse effects of Gcs, providing clinical applications for wide range of different diseases and disorders treated with Gcs.

### Materials and Methods

Chemicals. LY294002, Wortmannin (WM), AZD8055 were from LC Laboratories (Woburn, MA), Dexamethasone (Dex) and other chemicals were from Sigma-Aldrich (St. Louis, MO).

Murine osteocytes. MC3T3 murine preosteoblast cells were maintained in MEM alpha medium supplemented with 10% FBS. To induce preosteoblast differentiation to osteocytes, cells were incubated with the medium supplemented with 30 mM Beta-glycerophospate disodium salt hydrate (Sigma G-9422), and 50 mM L-ascorbic acid 2-phosphate sesquimagnesium salt hydrate (Sigma A8960)) for one week. Following the differentiation period, the cells were pre-treated with Rapamycin (1 uM) or vehicle (for control) for 6 hrs, and then treated with Dexamethasone (DEX, 1 uM) or vehicle for 24 h.

Computational screen of REDD1 inhibitors. Putative REDD1 inhibitors were identified by computational screening of LINCS library (Ref. 12). The molecular signature of each compound in each experiment is presented at LINCS as a list of DEGs—differentially expressed genes, ordered by descending expression fold-change. The top putative REDD1 inhibitors were selected according to the number of LINCS experiments in which REDD1 was within 100 most down-regulated genes in treated cells. For statistical computing, we used the R project version 3.2.5 (www.r-project.org/) was used. The top 20 REDD1 suppressors predicted by this method were sirolimus, wortmannin, alvocidib, AZD8055, triptolide, WYE-125132, OSI-027, CGP-60474, PI-103, BMS-387032, emetine, NVP-BEZ235, torin 1, LY294002, QL-X-138, and mitoxantrone.

Cell cultures. Acute lymphoblastic leukemia cell line CEM and mantle cell lymphoma cell line Granta were obtained from ATCC and cultured in RPMI-1640 (Thermo Fischer) with 10% FBS (HyClone), sodium piruvate (10 mM), HEPES (10 mM) and antibiotics (referred thereafter as complete medium; Thermo Fischer) (Ref 14; incorporated by reference in its entirety). Peripheral blood samples were collected from healthy volunteers. The ethical Committee of the Blokhin Cancer Research Center approved this study, written informed consents were provided. Mononuclear cell fraction was isolated by 1.077 g/ml Ficoll-Isopaque density-gradient centrifugation and cultured in the complete medium. Cells were pretreated with Rapa (0.1-5 uM), LY294002 (10-100 uM), WM (1-10 uM) and AZD8055 (0.1-2 uM) or vehicle for 6 h, then were treated for Dex (1-10 uM) or vehicle for 24-144 h as indicated.

Western blot analysis. Western blot analysis was performed (Ref 10; incorporated by reference in its entirety). The following antibodies were used: GR (H-300) (Santa Cruz Biotechnology, Dallas, TX), phospho-GR (Ser211), phospho-4E-BP1 (Thr37/46), phospho-Akt (Ser473), Akt, 4E-BP1, cleaved and full-length PARP, GAPDH, HDAC1 (Cell Signaling, San Jose, CA), REDD1 (Proteintech Group, Rosemont, IL).

Analysis of cell viability and apoptosis induction. Cell viability was assessed using the MTT assay as follows: cells were incubated at 37° C. for 3 h with 20 ul of MTT solution (5 mg/ml). Supernatant was discarded and 150 μL of 100% DMSO was added to each well. Optical density was measured at 495 nm using a microplate reader MultiScan MCC 340 (Labsystems). The IC50 values was calculated using Quest Graph IC50 Calculator free software. Isobolograms were constructed (Ref 15; incorporated by reference in its entirety). IC50 values of PI3K inhibitors or Dex as single agents were plotted as 1 on the x and y axes, respectively. Various concentrations of PI3K inhibitors and for Dex were combined. The IC50 values for each compound in the combination were calculated and plotted as a percentage of each single drug. The disposition of data points below, above or on the line of additivity indicates synergy, antagonism or additivity, correspondingly.

Apoptosis induction was evaluated using Western blot analysis of PARP cleavage and propidium iodine (PI) staining (Ref. 14; incorporated by reference in its entirety).

RNA extraction and Q-PCR. Total RNA isolation, reverse transcription and Q-PCR were performed (Ref 10; incorporated by reference in its entirety). Primers were designed with NCBI Primer-BLAST (Table 1). Results were normalized to the expression of the housekeeping RPL27 gene.

TABLE 1
Primer sets for Q-PCR analysis

| Gene symbol | | Primer sequence: |
|---|---|---|
| Mouse | Human | sense/antisense (5′-3′) |
| Rankl | | CAGCATCGCTCTGTTCCTGTA |
| | | (SEQ ID NO: 1) |
| | | CTGCGTTTTCATGGAGTCTCA |
| | | (SEQ ID NO: 2) |
| Opg | | ACCCAGAAACTGGTCATCAGC |
| | | (SEQ ID NO: 3) |
| | | CTGCAATACACACACTCATCACT |
| | | (SEQ ID NO: 4) |
| Rpl27 | | GCCCTGGTGGCTGGAATTGACC |
| | | (SEQ ID NO: 5) |
| | | TTGCGCTTCAAAGCTGGGTCCC |
| | | (SEQ ID NO: 6) |
| | CCND1 | CTACCTTCCGCAGTGCTCCTA |
| | | (SEQ ID NO: 7) |
| | | CCCAGCCAAGAAACGGTCC |
| | | (SEQ ID NO: 8) |
| | CCND2 | GCTGGAGCCCGTGAAAAAGA |
| | | (SEQ ID NO: 9) |
| | | CTCCGCCTCTGGCATTTTG |
| | | (SEQ ID NO: 10) |
| | CD86 | CTGCTCATCTATACACGGTTACC |
| | | (SEQ ID NO: 11) |
| | | GGAAACGTCGTACAGTTCTGTG |
| | | (SEQ ID NO: 12) |
| | FKBP51 | GAATGGTGAGGAAACGCCGAT |
| | | (SEQ ID NO: 13) |
| | | TGCCAAGACTAAAGACAAATGGT |
| | | (SEQ ID NO: 14) |
| | GILZ | AACACCGAAATGTATCAGACCC |
| | | (SEQ ID NO: 15) |
| | | TGTCCAGCTTAACGGAAACCA |
| | | (SEQ ID NO: 16) |
| | IL7R | CGTCTATCGGGAAGGAGCCAAT |
| | | (SEQ ID NO: 17) |
| | | GCTGGATAAATTCACATGCGTCCA |
| | | (SEQ ID NO: 18) |
| | KLF9 | GAAACACGCCTCCGAAAAGAGG |
| | | (SEQ ID NO: 19) |
| | | GAAAGGGCCGTTCACCTGTATG |
| | | (SEQ ID NO: 20) |
| | MKP1 | ACCACCACCGTGTTCAACTTC |
| | | (SEQ ID NO: 21) |
| | | TGGGAGAGGTCGTAATGGGG |
| | | (SEQ ID NO: 22) |
| | REDD1 | TAGCCTTTGGGACCGCTTCTCGT |
| | | (SEQ ID NO: 23) |
| | | CAGGTAAGCCGTGTCTTCCTCCG |
| | | (SEQ ID NO: 24) |
| | RPL27 | ACCGCTACCCCCGCAAAGTG |
| | | (SEQ ID NO: 25) |
| | | CCCGTCGGGCCTTGCGTTTA |
| | | (SEQ ID NO: 26) |

Luciferase assay. GRE.Luc, NF-κB.Luc and control mCMV.Luc reporter cells were generated (Ref. 13; incorporated by reference in its entirety) using viral stocks obtained from Northwestern University SDRC DNA/RNA delivery Core. Luciferase activity was measured (Ref. 10; incorporated by reference in its entirety).

Xenograft tumor growth study. 10 million Granta cells in 200 ul BD Matrigel Matrix were subcutaneously injected into the right flank of 7 weeks old female nu/nu mice (Taconic). Animals were randomized and treated every 48 h with LY294002 (20 mg/kg), Rapamycin (5 mg/kg) of vehicle (30% PEG3350, 4% DMSO, 5% Tween 20 in PBS) followed by Dex (1 mg/kg) of vehicle (5% DMSO in PBS) 6 h later. Body weight was recorded twice a week. Tumor size was measured twice a week by digital calipers, drug interaction mode was calculated based on combination index method (Refs. 16,17; incorporated by reference in their entireties). Combination index (CI) of Dex+Rapa/LY294002 was calculated as ratio (observed fraction value of combination group)/(expected fraction value of combination group). The observed fractions were calculated as ratio (mean value of experiment)/(mean value of control), and the expected fractions values of combination effect between combined groups and single drug groups were calculated as expected fraction=(mean observed fraction of Dex)×(mean observed fraction of Rapa/LY294002). CI<1, CI=1 and CI>1 indicates a synergy, additive effect and antagonism, respectively.

Glucocorticoid-induced osteoporosis (GIOP). GIOP was induced as described (18) by i.p. injection of Dex (10 mg/kg) in 12 weeks old BALB/c female mice (“Stolbovaya”, Moscow region, Russia) every 48 h for 5 weeks. 6 h prior to Dex, animals were treated i.p. with LY294002 (20 mg/kg), Rapamycin (5 mg/kg) of vehicle (30% PEG3350, 4% DMSO, 5% Tween-20 in PBS). Body weight was recorded twice a week.

All animal experiments were in compliance with Northwestern University ACUC and Blokhin NMRCO approved protocols.

Histological analysis and immunostaining. Xenograft and skin samples were fixed in formaldehyde, embedded in paraffin, sections were stained with hematoxylin and eosin. The bone samples after formaldehyde fixation were decalcinated in decalcination buffer (0.5 M EDTA, 0.7 mM NaOH, pH=7.0) for 14 days, then washed with PBS twice and further processed according to standard histology protocol. Ki67 and Caspase 3 were detected by immunostaining with antibodies to Ki-67 and Caspase-3 (Cell Signaling, San Jose, CA).

Morphometric analysis. Quantification of the epidermal width, dermal adipose thickness, bone thickness and cellularity of xenograft tissue was performed in sections stained with hematoxylin and eosin. At least 5 individual fields per slide of each individual sample/treatment (at least 50 images/treatment group) were analyzed using Axioplan2 microscope software (Carl Zeiss). The epidermal, dermal adipose, or bone thickness and cell density in treated animals is presented as % to control animals.

Statistical analysis. Mean and standard deviation values were calculated using Microsoft Excel software. The treatment effects in each experiment were compared by one-way ANOVA or t-test. Differences between groups were considered significant at P<0.05. All in vitro experiments were repeated three times. 10 animals/experimental group were used.

### Results

PI3K inhibitors suppressed REDD1 expression in lymphoid cells. Prior to experiments conducted herein, REDD1 pharmacological inhibitors were not known. Thus, inhibitors of REDD1 expression were searched for using bioinformatics screening of a large LINCS library of transcriptomic signatures of ˜20,000 small molecule drugs tested at large range of concentrations in ˜50 human cell lines (lincsproject.org/LINCS/). Compounds were scored according to the number of LINCS experiments in which REDD1 expression was significantly inhibited (REDD1 was within top 100 down-regulated genes), and found that inhibitors of the PI3K/mTOR/Akt pathway represented the major pharmacological class among the top inhibitors of REDD1 expression.

Three structurally-diverse exemplary compounds were selected for testing: classical experimental PI3K inhibitors Wortmannin (WM) and LY294002; and dual mTOR inhibitor AZD8055 that recently entered clinical trials as anti-cancer drug (Ref 19; incorporated by reference in its entirety). mTOR inhibitor, Rapamycin (Rapa), was also used as a previously characterized reference REDD1 inhibitor (Ref. 13; incorporated by reference in its entirety). PI3K activation leads to the phosphorylation and activation of Akt, which in turn activates mTOR in both mTORC1 and mTORC2 complexes (Ref 20; incorporated by reference in its entirety). Thus, it was expected that the selected inhibitors could affect to some degree all branches of PI3K/Akt/mTOR signaling.

The anti-REDD1 effects of these compounds were tested in mantle cell lymphoma Granta and acute lymphoblastic leukemia CEM cells, which are widely used for the screening of novel chemotherapeutics for blood cancer treatment (Refs. 1,2,14; incorporated by reference in their entireties).

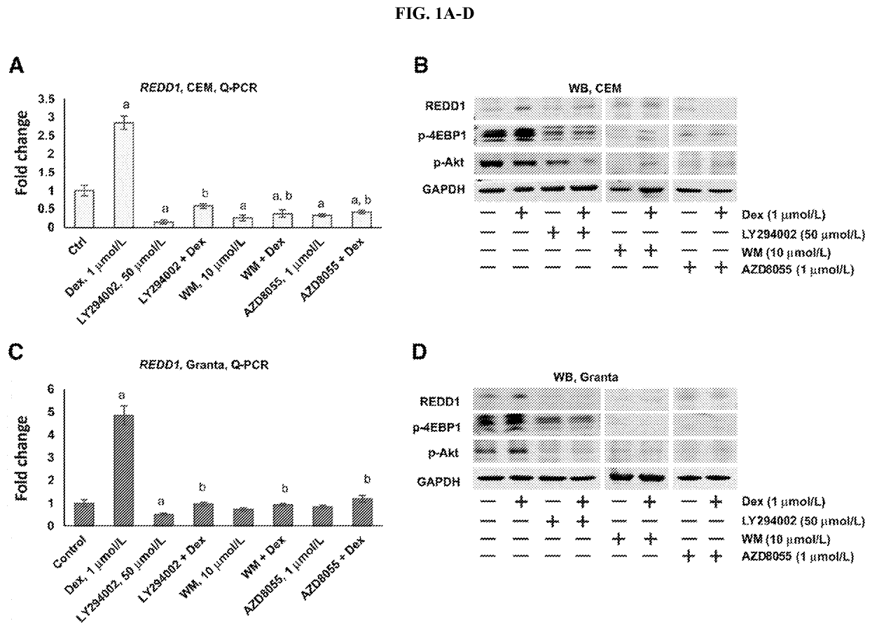

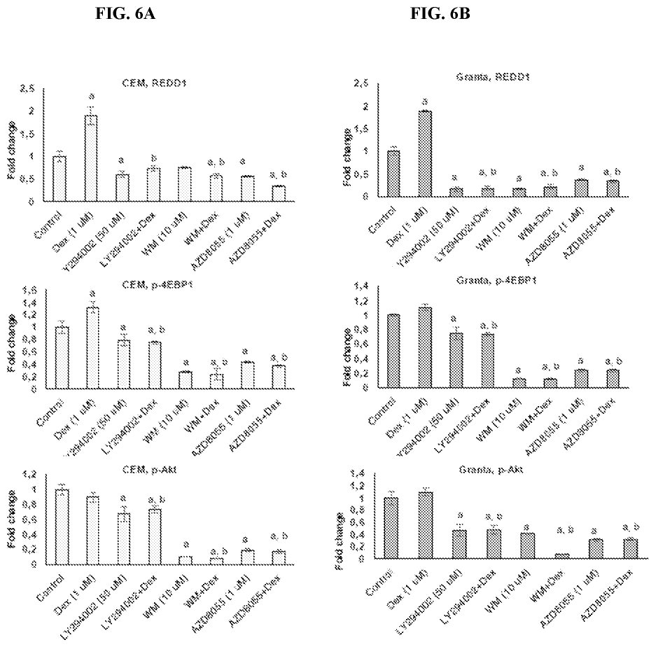

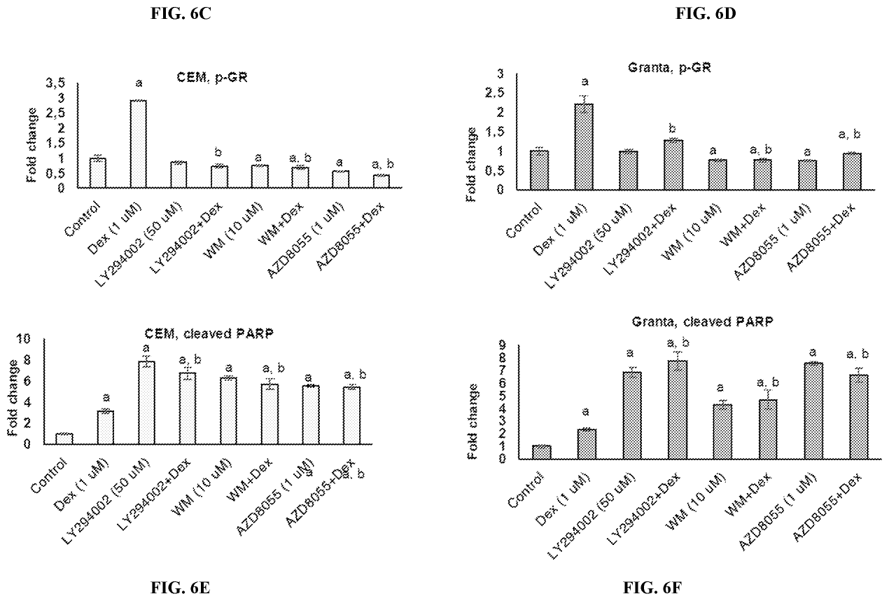

The optimal regimen of cell treatment: pre-treatment with REDD1 inhibitors for 6 h followed by Dex treatment for 24 h was chosen (Ref. 12,13; incorporated by reference in their entireties). Dex alone significantly induced REDD1 expression at mRNA/protein levels in both Granta and CEM cells. All tested compounds, LY294002 (50 uM), WM (10 uM) and AZD8055 (1 uM) prevented REDD1 induction (FIG. 1, FIG. 6).

The selected regimen and concentrations of REDD1 inhibitors strongly down-regulated PI3K/Akt/mTOR signaling monitored by phosphorylation of mTOR substrate 4E-BP1 (eukaryotic initiation factor 4E binding protein 1), and Akt at Ser473, the major site regulating Akt activity (FIG. 1 B, D). PI3K/Akt/mTOR inhibitors also significantly reduced basal expression of REDD1 mRNA (in CEM cells, FIG. 1A), and protein (more pronounced in Granta cells, FIG. 1D).

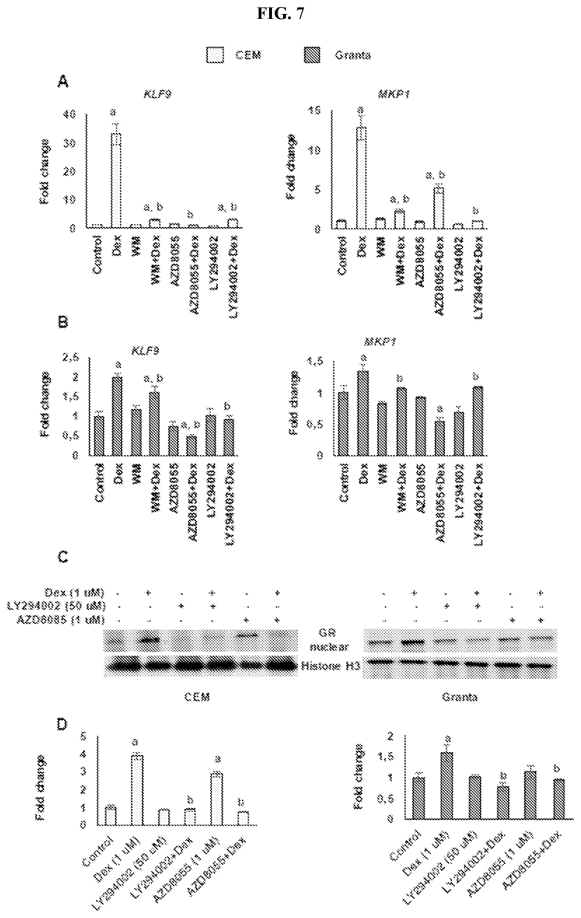

PI3K inhibitors decreased GR phosphorylation, nuclear translocation, and shifted GR function towards transrepression. REDD1 is a GR target gene (Refs. 10,21; incorporated by reference in their entireties). To assess whether REDD1 inhibitors could modify GR function, LY294002, WM and AZD8055 effect on GR TA and TR was evaluated using CEM and Granta cells transduced with GRE.Luc and NF-kB.Luc reporters. All three tested compounds inhibited Dex-induced GRE. Luciferase activity in both cell lines (FIG. 2A, C). Moreover, they also negatively affected basal GR activity when applied individually, with LY294002 being the most effective. Attenuation of GR TA was further confirmed by Q-PCR analysis of known GR-target genes including GR chaperone FKBP51 (Refs. 12,14; incorporated by reference in their entireties), mediator of Gcs anti-inflammatory effects GILZ (22,23), transcriptional activator KLF9 (24) and inhibitor of cytokine production MKP-1 (25) (FIG. 2B, D and FIG. 7A, B).

NF-kB activity was strongly reduced according to kB.Luciferase test by Dex as well as by PI3K/Akt/mTOR inhibitors during individual treatment, and in case of Dex combination with AZD8055 or WM, additive inhibitory effect in both cell lines was observed (FIG. 2E, G). This finding was further corroborated by Q-PCR analysis of negative regulation of several endogenous genes such as cell cycle regulators cyclins CCND1/D2, and IL7R involved in lymphoid cell survival (Ref. 26; incorporated by reference in its entirety), and to the lesser extent, a marker of malignant lymphoid cells CD86 (FIG. 2F, H).

To identify the molecular mechanisms underlying GR functional changes induced by PI3K/Akt/mTOR inhibitors, the effect of these compounds on major steps in GR activation was assessed: GR phosphorylation at Ser211 and nuclear translocation that are critical for GR TA (Refs. 27,28; incorporated by reference in their entireties).

All studied compounds prevented GR phosphorylation induced by Dex in both cell types to a different degree (FIG. 2I). Two out of 3 tested PI3K inhibitors, LY294002 and AZD8055, in addition to the effect on GR phosphorylation significantly prevented GR nuclear import (FIG. 7C).

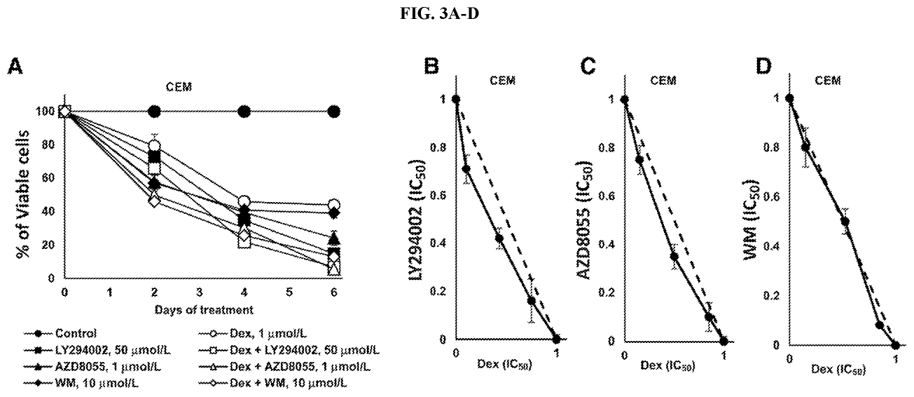

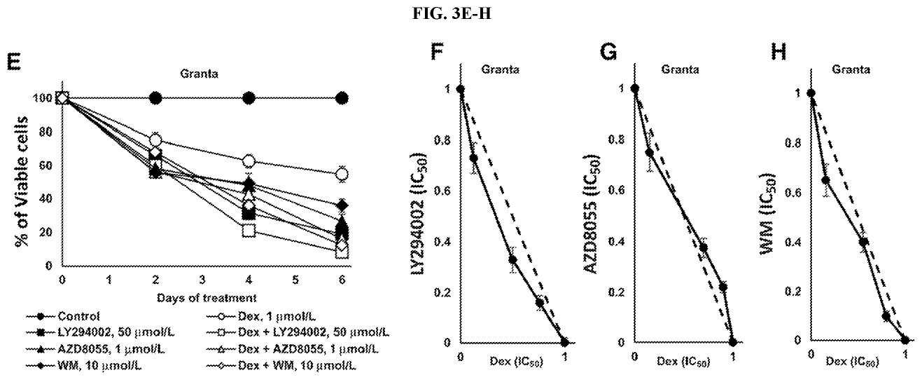

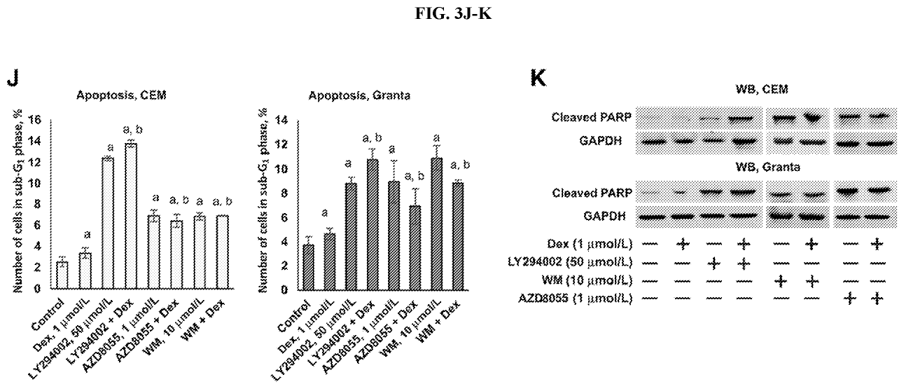

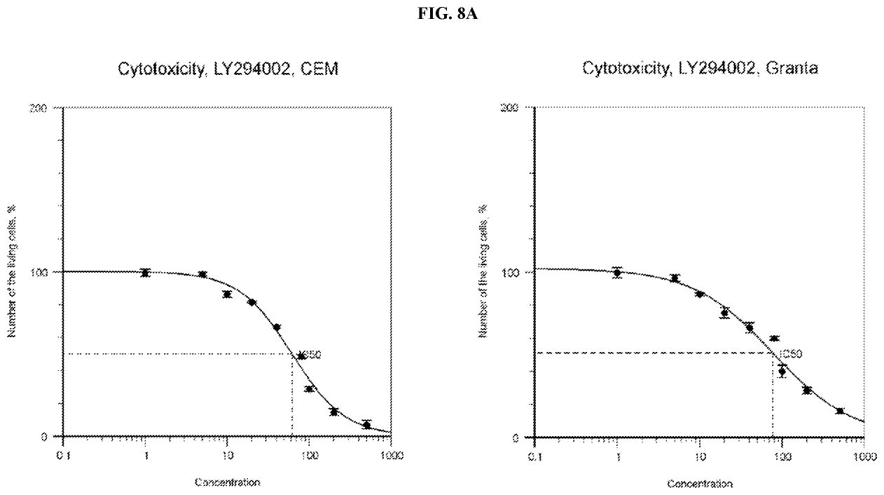

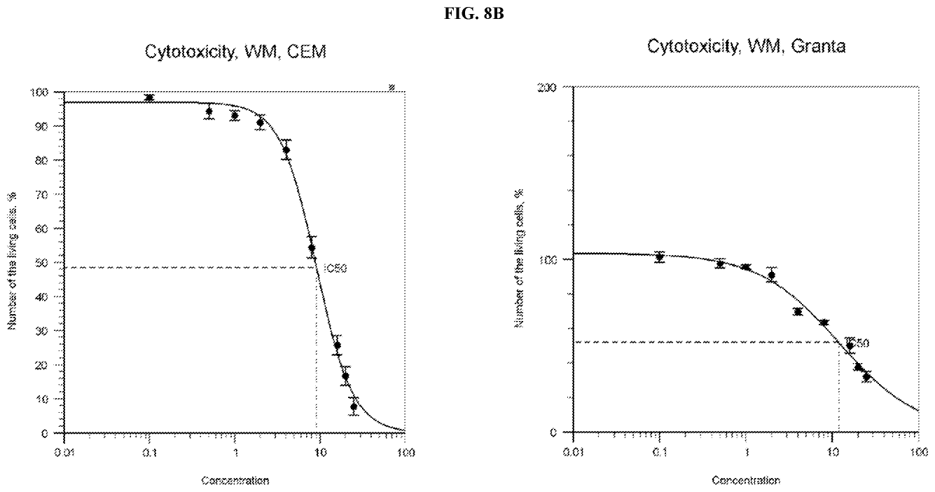

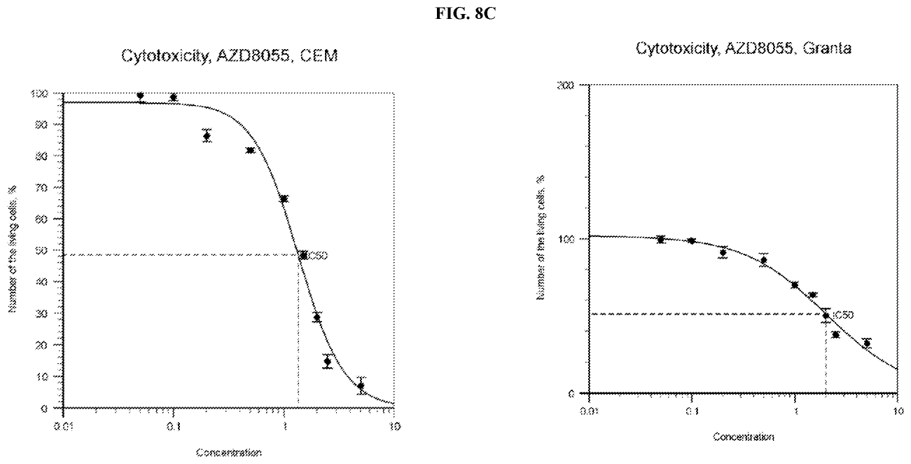

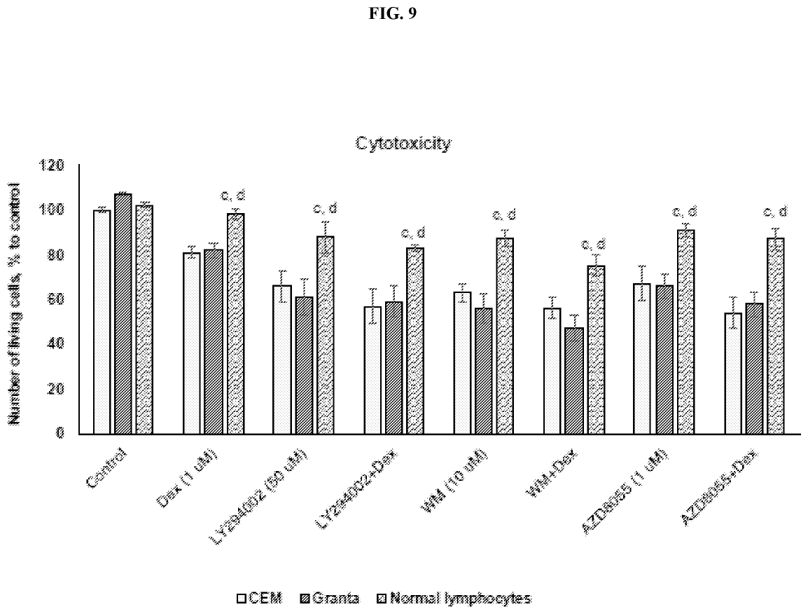

Cooperative anti-lymphoma effects of Dex and PI3K/Akt/mTOR inhibitors in vitro. PI3K/Akt/mTOR inhibitors were developed as anti-cancer drugs. To assess whether and how PI3K/Akt/mTOR inhibitors interacted with Dex anti-lymphoma effects, experiments were conducted to evaluate the efficacy of the combined treatment in vitro using such readouts as cell viability (MTT test) and apoptosis (PARP cleavage and sub-G1-phase cell population determined by FACS). The cytotoxic effect of Dex and PI3K inhibitors in wide concentration range (LY294002 1-500 uM, WM 0.1-25 uM, AZD8055 0.05-5 uM) was assessed (FIG. 8) and determined IC50 values after 24 h treatment (Table 8). PI3K inhibitors at optimal, close to IC50 concentrations (50 uM for LY294002, 1 uM for AZD8055, and 10 uM for WM) decreased CEM and Granta survival by 70-90%, and Dex (1 uM) by 50-55% when used alone for 6 days (FIG. 3A, E). At the same time, normal lymphocytes from healthy volunteers were significantly more resistant to cytotoxic effects of combinational treatments (FIG. 9).

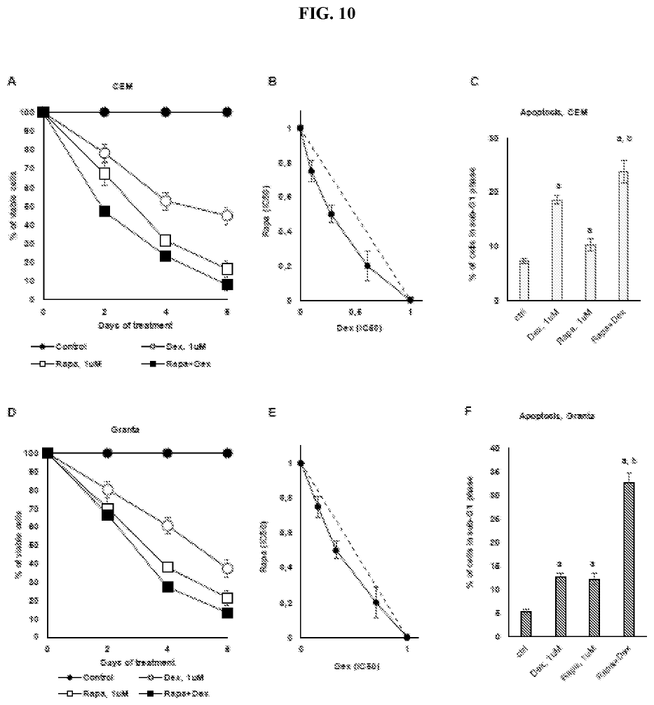

To assess the type of interactions between Dex and PI3K/Akt/mTOR inhibitors in anti-lymphoma tests, determined IC50 values for inhibitors and Dex after 24 h treatment were used Table 2). Isobolograms were generated and statistical models were used to determine synergy, additivity or antagonism between Dex and PI3K inhibitors (FIG. 3B-D, F-H). Isobologram analysis revealed synergy in antiproliferative effects of Dex and LY294002 in both cell types (FIG. 3B, F). Synergy was also determined for Dex and Rapa, used as reference REDD1 inhibitor (FIG. 10). The effects of Dex and AZD8055 or WM on lymphoma cell survival were mostly additive (FIG. 3C, D, G, H).

TABLE 2
Cytotoxicity of WM, LY294002, AZD8055

| | IC50, | IC50, |
|---|---|---|
| Compound | CEM, uM | Granta, uM |
| Wortmannin | 9.145 | 11.944 |
| LY294002 | 61,969 | 77.588 |
| AZD8055 | 1,358 | 2,015 |

The effect of Dex combined with LY294002, AZD8055, WM or reference inhibitor Rapa was examined on apoptosis. When used alone, Dex and all three PI3K inhibitors as well as Rapa stimulated apoptosis in CEM and Granta cells as assessed by increase in sub-G1-phase cell population (FIG. 3J and FIG. 10). LY294002 cooperated with Dex in apoptosis induction; other inhibitors increased apoptosis development compared to Dex only in both cell types. These findings were confirmed by Western blot analysis of PARP cleavage (FIG. 3K), that revealed the especially pronounced cooperative pro-apoptotic effects between LY294002 and Dex in CEM cells.

PI3K inhibitors enhance anti-lymphoma effects of Dexamethasone in vivo. To assess whether REDD1 inhibitors from PI3K/Akt/mTOR pharmacological class enhance Dex anti-lymphoma activity in vivo, previously established s.c. Granta xenograft model in athymic nu/nu female mice was used (Ref. 2; incorporated by reference in its entirety). LY294002 that displayed synergistic anti-lymphoma effects with Dex in vitro was selected. As a positive control, mTOR inhibitor Rapamycin, whose synergistic anti-cancer effects with Dex in vitro were reported (Ref 29; incorporated by reference in its entirety), was used.

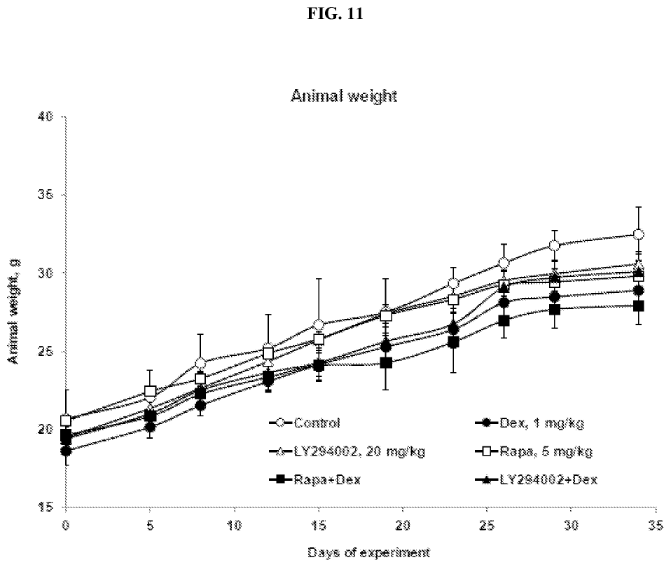

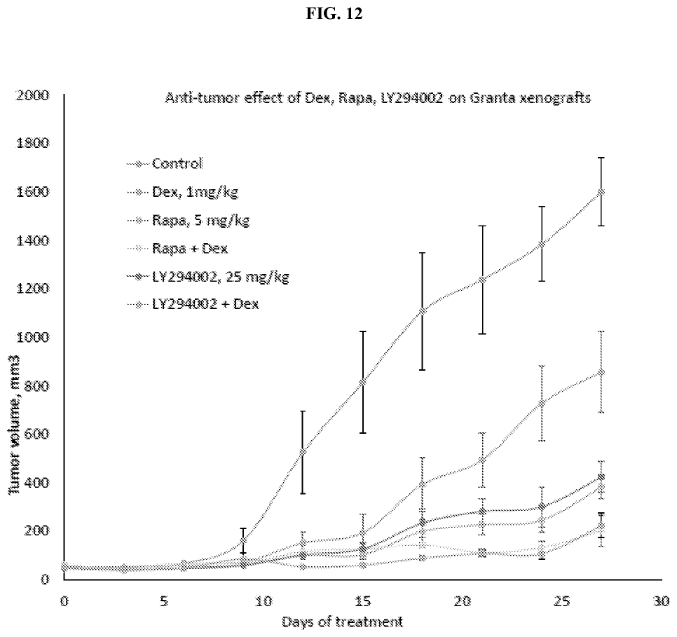

Animals were randomized to different treatment groups after tumor volume reached 50 mm3. All compounds were delivered via i.p. injections of LY29400 (20 mg/kg), Rapa (5 mg/kg) or vehicle in the morning followed by Dex treatment (1 mg/kg) or vehicle 6 h later following the optimal regimens for REDD1 inhibition developed in vitro (2,13). All treatment regiments were only minimally toxic according to the steady increase in body weight monitored twice weekly (FIG. 11). Tumor volume was measured also twice a week (FIG. 12).

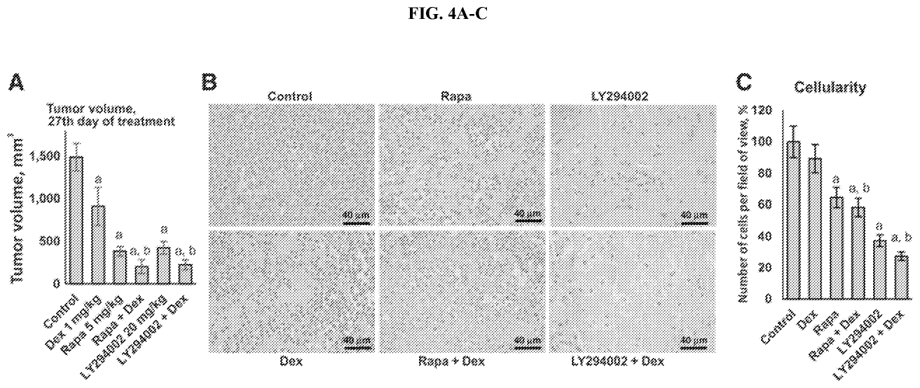

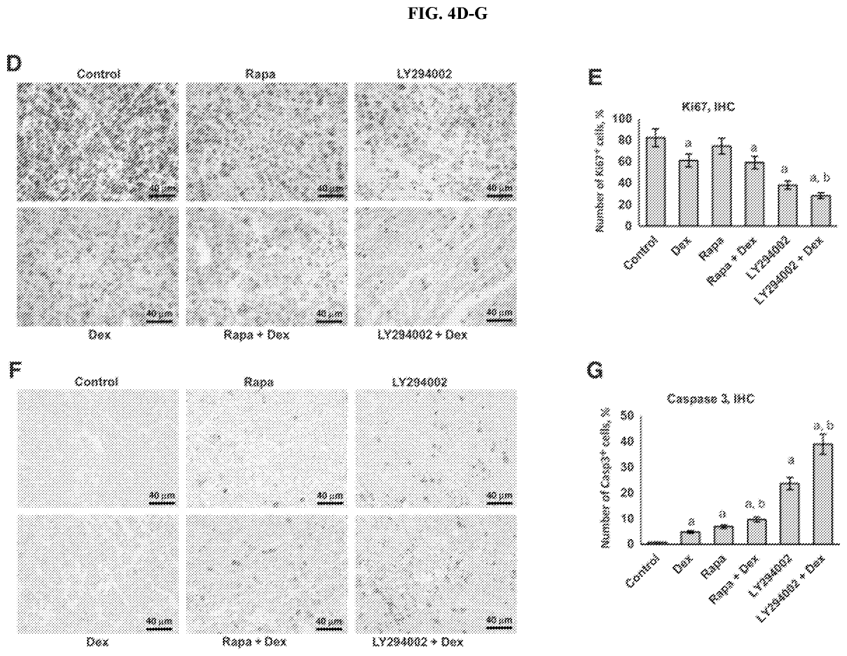

At the end-point of experiment (27^{th} day of treatment), the average tumor volume in control was 1489±161.3 mm3. In groups treated with Dex and LY294002 individually, the tumor volume decreased by ˜40% and ˜70% correspondingly (FIG. 4A). A remarkable 85% decrease in tumor volume was observed in the LY294002+Dex group. Similar cooperation in anti-cancer effects was observed for Rapa and Dex (˜75% decrease in tumor volume in Rapa-treated group and ˜85% in Rapa+Dex group). To determine the mode of combined action of Dex and LY294002/Rapa, the combination index (CI) approach was used, based on the difference between the expected and observed mean tumor volumes in Dex+LY294002 or Dex+Rapa groups compared to control. Calculated CI values of 0.87 for Dex+LY294002 and 0.85 for Dex+Rapa indicated synergistic mode of interaction.

The reduction in xenograft tumor volume in Dex+LY294002 and Dex+Rapa groups reflected drastic decrease of tumor cellularity, by 50-70% in groups with combined treatments with superior effect of Dex+LY294002 (FIG. 4B, C). It is known that the major effects of Gcs in transformed lymphoid cells are growth inhibition and apoptosis (Refs. 33,34; incorporated by reference in their entireties). Thus, proliferative activity of the cells in xenografts was evaluated, as well as apoptosis by immunostaining with proliferation marker Ki67 and apoptosis marker caspase 3. The lowest number of Ki67-positive cells and the highest percentage of apoptotic cells was observed in the xenografts from LY294002+Dex treated mice (FIG. 4D-G) that further confirmed that LY294009 co-treatment enhanced anti-lymphoma effects of Dex. Effects of Rapa on antiproliferative and proapoptotic Dex activity were statistically significant for increased apotosis induction.

Protective effect of LY294002 and Rapamycin on systemic atrophic effects of Dex in osteoporosis and skin atrophy models. LY294002 displayed synergistic anti-lymphoma effects with Dex both in vitro and in vivo. Its tissue protective effect against atrophy induced by systemic exposure to Dex was tested. The mechanisms of Gcs atrophic effects are complex, and depend on the tissue. In skin and muscle, the GR molecular signature appeared to be similar (Ref. 10; incorporated by reference in its entirety), and in both tissues Gcs significantly shift metabolism to catabolic processes that affect lipids, sphingolipids, proteins (Refs. 10,35; incorporated by reference in their entireties). The steroid-induced skin atrophy involves all skin compartments and results in overall skin thinning, changes in collagen synthesis, atrophy of epidermis and dermal adipose (Refs. 8,36,37; incorporated by reference in their entireties).

In bone, Gcs induce the shift of bone cell populations towards bone-resorbing osteoclasts via increased osteoclast formation and inhibition of proliferation of bone-forming osteoblasts leading to an increased risk of fragility and bone fractures (Ref. 38; incorporated by reference in its entirety). One of the major signaling pathways that regulates osteoclast formation from their precursors as well as their activation and survival during normal bone remodeling and in a variety of pathologic conditions is mediated by RANK (receptor activator of nuclear factor κB)/RANKL (RANK ligand) signaling (Ref. 39; incorporated by reference in its entirety). Another important regulator of osteoclasts is cytokine Osteoprotegerin (OPG) that acts as a decoy receptor for RANKL. Thus, OPG protects the skeleton from excessive bone resorption, and RANKL/OPG ratio is frequently used as an important determinant of bone mass and skeletal integrity (Ref. 39; incorporated by reference in its entirety). Collagen family proteins (especially Col1a1 whose polymorphisms are associated with age-related osteoporosis) also play a key role in bone formation, structure and mineralization (Refs. 40,41; incorporated by reference in their entireties).

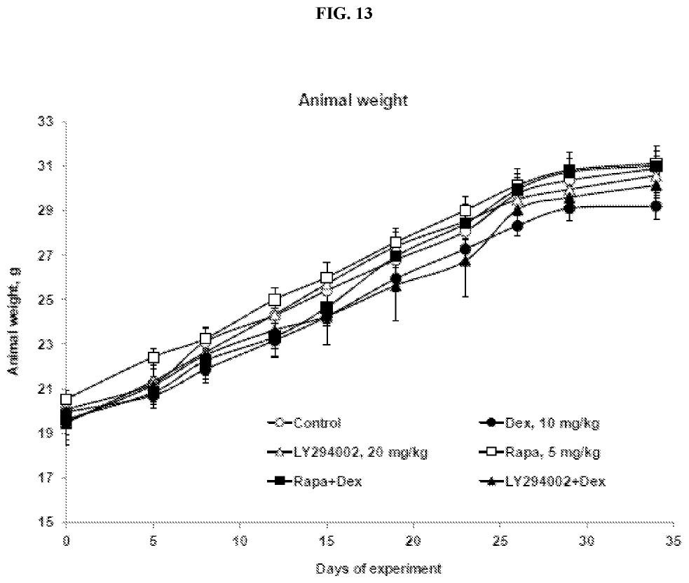

To test whether REDD1 inhibitors protect against systemic atrophic effects of Gcs, a model of Gcs-induced osteoporosis in Balb/c female mice was used (18) to assess atrophy in both bone and skin induced by high-dose i.p. Dex injections (10 mg/kg) every 48 h for 5 weeks. LY294002 (20 mg/kg), Rapa (5 mg/kg) or vehicle were injected 6 h prior animal treatment with Dex. Over the course of the experiment general animal health, as determined by cage-side observations and animal body weight, was similar between control and experimental groups; moreover, we saw a normal steady increase in animal body weight in all groups during the experiment (FIG. 13).

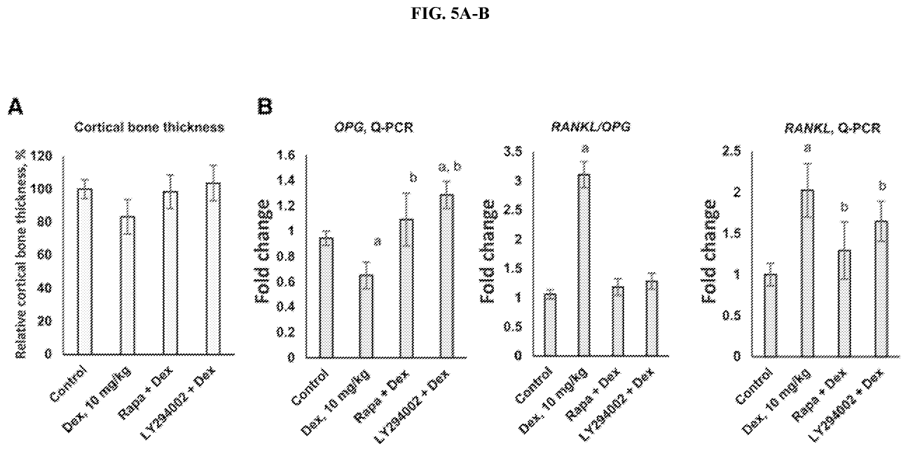

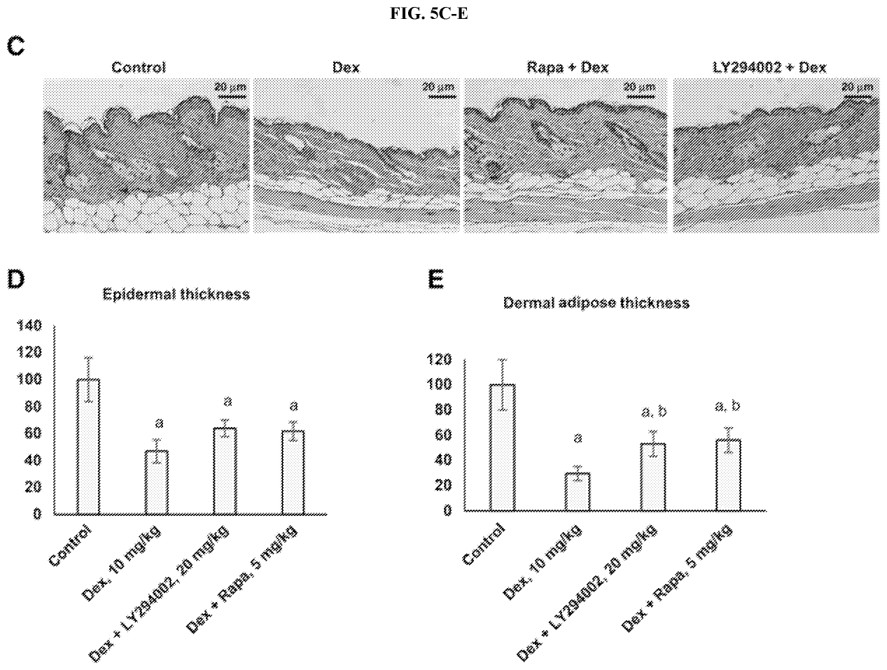

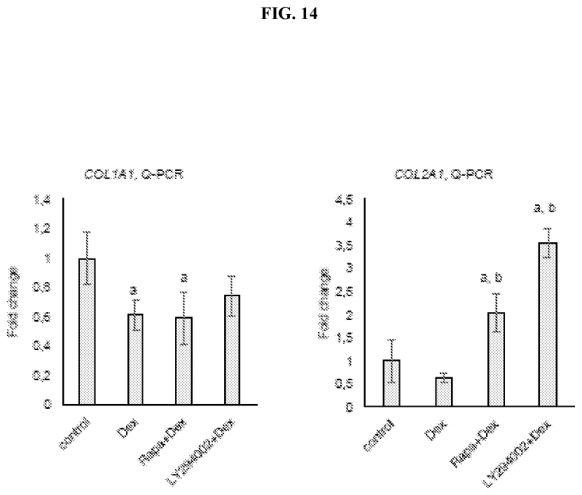

The tendency in decrease of femoral bone thickness between control and Dex-treated animals was observed (FIG. 5A). Changes in widely accepted osteoporosis markers: RANKL (associated with bone resorption), OPG (protector from bone resorption) and RANKL/OPG ratio clearly indicated that in Dex-treated group femur bones started to be affected by osteoporosis: RANKL/OPG ratio was significantly increased after 5-week treatment (FIG. 5B). Both LY294002 and Rapa induced down-regulation of RANKL expression, up-regulation of OPG expression, and, correspondingly, the significant decrease of RANKL/OPG ratio towards control level. Moreover, expression of Col1a1 and especially Col2a1 decreased after Dex chronic treatment, was restored in bone samples from animals treated with LY294002 or Rapa (FIG. 14).

To test whether LY294002 and Rapamycin could prevent development of skin atrophy induced by systemic Dex delivery, morphological changes in skin of Balb/c female mice were assessed. Chronic treatment with systemic Dex led to a very significant cutaneous atrophy: epidermal thickness was reduced by ˜50%, and dermal adipose tissue was reduced by ˜ 70% (FIG. 5C-E). LY294002 and Rapamycin partially protected skin from these hypoplastic effects of Dex: LY294002+Dex-treated skin displayed only 35% and 45% reduction in epidermal and dermal adipose thickness accordingly. The protective effects of Rapamycin were similar.

### Example 2

### Rapamycin Prevents Induction of Atrophogene REDD1 by Dexamethasone in Murine Osteocytes

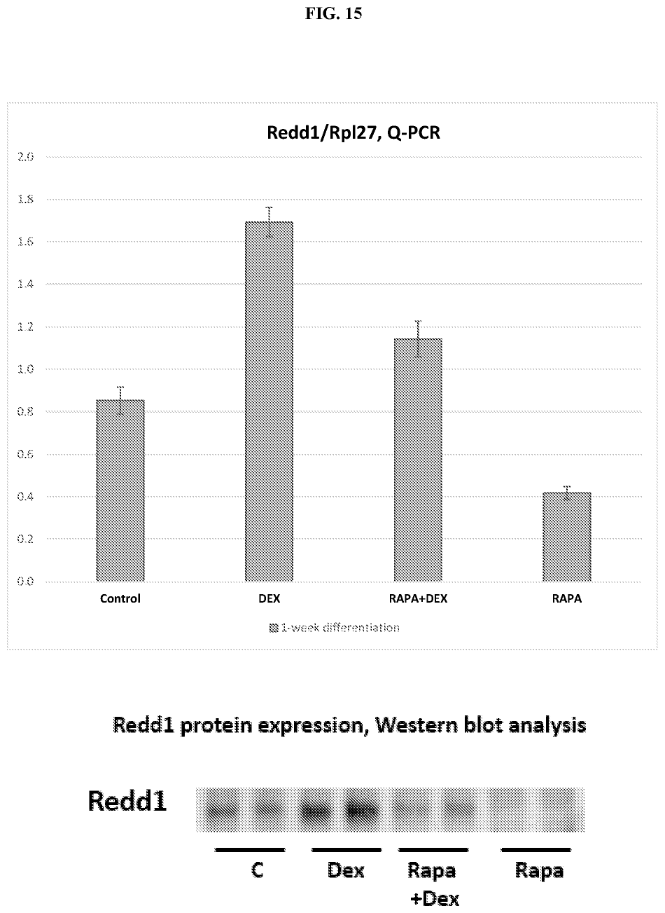

MC3T3 murine preosteoblast cells were grown in MEM alpha medium supplemented with 10% FBS till confluency, and then were placed in the differentiating medium (MEM alpha medium with 10% FBS, supplemented with 30 mM Beta-glycerophospate disodium salt hydrate (Sigma G-9422), and 50 mM L-ascorbic acid 2-phosphate sesquimagnesium salt hydrate (Sigma A8960)) for one week. Following the differentiation period, the cells were pre-treated with Rapamycin (RAPA, 1 uM) or vehicle (for control) for 6 hrs, and then treated with Dexamethasone (DEX) or vehicle for 24 h. The expression of REDD1 was determined by Western Blot and Q-PCR and normalized to Rp127 expression (FIG. 15).

### REFERENCES

The following references, some of which are cited above by number, are herein incorporated by reference in their entireties.
1. Scheijen B. Molecular mechanisms contributing to glucocorticoid resistance in lymphoid malignancies. Cancer Drug Resist. 2019; 2:647-664.
2. Lesovaya E, Yemelyanov A, Swart A C, Swart P, Haegeman G, Budunova I. Discovery of Compound A—a selective activator of the glucocorticoid receptor with anti-inflammatory and anti-cancer activity. Oncotarget. 2015; 6:30730-44.
3. Hachemi Y, Rapp A E, Picke A-K, Weidinger G, Ignatius A, Tuckermann J. Molecular mechanisms of glucocorticoids on skeleton and bone regeneration after fracture. J Mol Endocrinol. 2018; 61:R75-90.
4. Conaway H H, Henning P, Lie A, Tuckermann J, Lerner U H. Activation of dimeric glucocorticoid receptors in osteoclast progenitors potentiates RANKL induced mature osteoclast bone resorbing activity. Bone. 2016; 93:43-54.
5. Kleiman A, Tuckermann J P. Glucocorticoid receptor action in beneficial and side effects of steroid therapy: lessons from conditional knockout mice. Mol Cell Endocrinol. 2007; 275:98-108.
6. Ramamoorthy S, Cidlowski J A. Ligand-induced repression of the glucocorticoid receptor gene is mediated by an NCoR1 repression complex formed by long-range chromatin interactions with intragenic glucocorticoid response elements. Mol Cell Biol. 2013; 33:1711-22.
7. De Bosscher K, Vanden Berghe W, Haegeman G. The interplay between the glucocorticoid receptor and nuclear factor-kappaB or activator protein-1: molecular mechanisms for gene repression. Endocr Rev. 2003; 24:488-522.
8. Schoepe S, Schacke H, May E, Asadullah K. Glucocorticoid therapy-induced skin atrophy. Exp Dermatol. 2006; 15:406-20.
9. De Bosscher K, Beck I M, Haegeman G. Classic glucocorticoids versus non-steroidal glucocorticoid receptor modulators: survival of the fittest regulator of the immune system? Brain Behav Immun. 2010; 24:1035-42.
10. Baida G, Bhalla P, Kirsanov K, Lesovaya E, Yakubovskaya M, Yuen K, et al. REDD1 functions at the crossroads between the therapeutic and adverse effects of topical glucocorticoids. EMBO Mol Med. 2015; 7:42-58.
11. Britto F A, Begue G, Rossano B, Docquier A, Vernus B, Sar C, et al. REDD1 deletion prevents dexamethasone-induced skeletal muscle atrophy. Am J Physiol Endocrinol Metab. 2014; 307:E983-93.
12. Agarwal S, Mirzoeva S, Readhead B, Dudley J T, Budunova I. PI3K inhibitors protect against glucocorticoid-induced skin atrophy. EBioMedicine. 2019; 41:526-37.
13. Lesovaya E, Agarwal S, Readhead B, Vinokour E, Baida G, Bhalla P, et al. Rapamycin Modulates Glucocorticoid Receptor Function, Blocks Atrophogene REDD1, and Protects Skin from Steroid Atrophy. J Invest Dermatol. 2018; 138:1935-44.
14. Lesovaya E, Yemelyanov A, Kirsanov K, Popa A, Belitsky G, Yakubovskaya M, et al. Combination of a selective activator of the glucocorticoid receptor Compound A with a proteasome inhibitor as a novel strategy for chemotherapy of hematologic malignancies. Cell Cycle. 2013; 12:133-44.
15. Lin C-J, Lee C-C, Shih Y-L, Lin T-Y, Wang S-H, Lin Y-F, et al. Resveratrol enhances the therapeutic effect of temozolomide against malignant glioma in vitro and in vivo by inhibiting autophagy. Free Radic Biol Med. 2012; 52:377-91.
16. Chou T-C. Theoretical basis, experimental design, and computerized simulation of synergism and antagonism in drug combination studies. Pharmacol Rev. 2006; 58:621-81.
17. Yang H, Wang J, Fan J-H, Zhang Y-Q, Zhao J-X, Dai X-J, et al. Ilexgenin A exerts anti-inflammation and anti-angiogenesis effects through inhibition of STAT3 and PI3K pathways and exhibits synergistic effects with Sorafenib on hepatoma growth. Toxicol Appl Pharmacol. 2017; 315:90-101.
18. McLaughlin F, Mackintosh J, Hayes B P, McLaren A, Uings I J, Salmon P, et al. Glucocorticoid-induced osteopenia in the mouse as assessed by histomorphometry, microcomputed tomography, and biochemical markers. Bone. 2002; 30:924-30.
19. Naing A, Aghajanian C, Raymond E, Olmos D, Schwartz G, Oelmann E, et al. Safety, tolerability, pharmacokinetics and pharmacodynamics of AZD8055 in advanced solid tumours and lymphoma. Br J Cancer. 2012; 107:1093-9.
20. Dennis M D, Coleman C S, Berg A, Jefferson L S, Kimball S R. REDD1 enhances protein phosphatase 2A-mediated dephosphorylation of Akt to repress mTORC1 signaling. Sci Signal. 2014; 7:ra68.
21. Wang H, Kubica N, Ellisen L W, Jefferson L S, Kimball S R. Dexamethasone represses signaling through the mammalian target of rapamycin in muscle cells by enhancing expression of REDD1. J Biol Chem. 2006; 281:39128-34.
22. Yang N, Baban B, Isales C M, Shi X-M. Role of glucocorticoid-induced leucine zipper (GILZ) in inflammatory bone loss. PLoS One. 2017; 12:e0181133.
23. Fan H, Kao W, Yang Y H, Gu R, Harris J, Fingerle-Rowson G, et al. Macrophage migration inhibitory factor inhibits the antiinflammatory effects of glucocorticoids via glucocorticoid-induced leucine zipper. Arthritis Rheumatol (Hoboken, NJ). 2014; 66:2059-70.
24. Leigh R, Mostafa M M, King E M, Rider C F, Shah S, Dumonceaux C, et al. An inhaled dose of budesonide induces genes involved in transcription and signaling in the human airways: enhancement of anti- and proinflammatory effector genes. Pharmacol Res Perspect. 2016; 4:e00243.
25. Vollmer T R, Stockhausen A, Zhang J-Z. Anti-inflammatory effects of mapracorat, a novel selective glucocorticoid receptor agonist, is partially mediated by MAP kinase phosphatase-1 (MKP-1). J Biol Chem. 2012; 287:35212-21.
26. Ligons D L, Tuncer C, Linowes B A, Akcay I M, Kurtulus S, Deniz E, et al. CD8 lineage-specific regulation of interleukin-7 receptor expression by the transcriptional repressor Gfil. J Biol Chem. 2012; 287:34386-99.
27. Chen W, Dang T, Blind R D, Wang Z, Cavasotto C N, Hittelman A B, et al. Glucocorticoid receptor phosphorylation differentially affects target gene expression. Mol Endocrinol. 2008; 22:1754-66.
28. Galliher-Beckley A J, Cidlowski J A. Emerging roles of glucocorticoid receptor phosphorylation in modulating glucocorticoid hormone action in health and disease. IUBMB Life. 2009; 61:979-86.
29. Zhang C, Ryu Y-K, Chen T Z, Hall C P, Webster D R, Kang M H. Synergistic activity of rapamycin and dexamethasone in vitro and in vivo in acute lymphoblastic leukemia via cell-cycle arrest and apoptosis. Leuk Res. 2012; 36:342-9.
30. Wei G, Twomey D, Lamb J, Schlis K, Agarwal J, Stam R W, et al. Gene expression-based chemical genomics identifies rapamycin as a modulator of MCL1 and glucocorticoid resistance. Cancer Cell. 2006; 10:331-42.
31. Hall C P, Reynolds C P, Kang M H. Modulation of Glucocorticoid Resistance in Pediatric T-cell Acute Lymphoblastic Leukemia by Increasing BIM Expression with the PI3K/mTOR Inhibitor BEZ235. Clin Cancer Res. 2016; 22:621-32.
32. Silveira A B, Laranjeira ABA, Rodrigues GOL, Leal P C, Cardoso B A, Barata J T, et al. PI3K inhibition synergizes with glucocorticoids but antagonizes with methotrexate in T-cell acute lymphoblastic leukemia. Oncotarget. 2015; 6:13105-18.
33. Ploner C, Schmidt S, Presul E, Renner K, Schrocksnadel K, Rainer J, et al. Glucocorticoid-induced apoptosis and glucocorticoid resistance in acute lymphoblastic leukemia. J Steroid Biochem Mol Biol. 2005; 93:153-60.
34. Amaral J D, Soli S, Steer C J, Rodrigues CMP. Role of nuclear steroid receptors in apoptosis. Curr Med Chem. 2009; 16:3886-902.
35. Shimizu N, Yoshikawa N, Ito N, Maruyama T, Suzuki Y, Takeda S, et al. Crosstalk between glucocorticoid receptor and nutritional sensor mTOR in skeletal muscle. Cell Metab. 2011; 13:170-82.
36. Hengge U R, Ruzicka T, Schwartz R A, Cork M J. Adverse effects of topical glucocorticosteroids. J Am Acad Dermatol. 2006; 54:1-15; quiz 16-8.
37. Kimura T, Doi K. Dorsal skin reactions of hairless dogs to topical treatment with corticosteroids. Toxicol Pathol. 27:528-35.
38. Frenkel B, White W, Tuckermann J. Glucocorticoid-Induced Osteoporosis. Adv Exp Med Biol. 2015; 872:179-215.
39. Boyce B F, Xing L. Biology of RANK, RANKL, and osteoprotegerin. Arthritis Res Ther. 2007; 9 Suppl 1:S1.
40. Clancy B M, Johnson J D, Lambert A J, Rezvankhah S, Wong A, Resmini C, et al. A gene expression profile for endochondral bone formation: oligonucleotide microarrays establish novel connections between known genes and BMP-2-induced bone formation in mouse quadriceps. Bone. 2003; 33:46-63.
41. Boskey A L, Imbert L. Bone quality changes associated with aging and disease: a review. Ann NY Acad Sci. 2017; 1410:93-106.
42. Petta I, Peene I, Elewaut D, Vereecke L, De Bosscher K. Risks and benefits of corticosteroids in arthritic diseases in the clinic. Biochem Pharmacol. 2019; 165:112-25.
43. Tan C Y, Hagen T. mTORC1 dependent regulation of REDD1 protein stability. PLoS One. 2013; 8:e63970.
44. Ismaili N, Garabedian M J. Modulation of glucocorticoid receptor function via phosphorylation. Ann NY Acad Sci. 2004; 1024:86-101.
45. Sacta M A, Tharmalingam B, Coppo M, Rollins D A, Deochand D K, Benjamin B, et al. Gene-specific mechanisms direct glucocorticoid-receptor-driven repression of inflammatory response genes in macrophages. Elife. 2018; 7.
46. Sacta M A, Chinenov Y, Rogatsky I. Glucocorticoid Signaling: An Update from a Genomic Perspective. Annu Rev Physiol. 2016; 78:155-80.
47. Dai J, Zhou S, Ge Q, Qin J, Li J, Ju H, et al. Recruitment of Brd3 and Brd4 to acetylated chromatin is essential for proinflammatory cytokine-induced matrix-degrading enzyme expression. J Orthop Surg Res. 2019; 14:59.
48. Habib T, Sadoun A, Nader N, Suzuki S, Liu W, Jithesh P V, et al. AKT1 has dual actions on the glucocorticoid receptor by cooperating with 14-3-3. Mol Cell Endocrinol. 2017; 439:431-43.
49. Polak R, Buitenhuis M. The PI3K/PKB signaling module as key regulator of hematopoiesis: implications for therapeutic strategies in leukemia. Blood. 2012; 119:911-23.
50. Curran E, Smith S M. Phosphoinositide 3-kinase inhibitors in lymphoma. Curr Opin Oncol. 2014; 26:469-75.
51. Molitoris J K, McColl K S, Swerdlow S, Matsuyama M, Lam M, Finkel T H, et al. Glucocorticoid elevation of dexamethasone-induced gene 2 (Dig2/RTP801/REDD1) protein mediates autophagy in lymphocytes. J Biol Chem. 2011; 286:30181-9.
52. Corsello S M, Bittker J A, Liu Z, Gould J, McCarren P, Hirschman J E, et al. The Drug Repurposing Hub: a next-generation drug library and information resource. Nat Med. 2017; 23:405-8.
53. Adcock I M, Mumby S. Glucocorticoids. Handb Exp Pharmacol. 2017; 237:171-96.
54. Robertson S, Allie-Reid F, Vanden Berghe W, Visser K, Binder A, Africander D, et al. Abrogation of glucocorticoid receptor dimerization correlates with dissociated glucocorticoid behavior of compound a. J Biol Chem. 2010; 285:8061-75.

1. A method of treating, preventing, and/or reducing glucocorticoid-induced side-effects in a subject comprising administering to the subject a PI3K/Akt/mTOR inhibitor.

2. The method of claim 1, wherein the PI3K/Akt/mTOR inhibitor is co-administered with the glucocorticoid.

3. The method of claim 1, wherein the PI3K/Akt/mTOR inhibitor is administered to a subject that has previously been administered the glucocorticoid.

4. The method of claim 1, wherein the subject exhibits glucocorticoid-induced side-effects.

5. The method of claim 1, wherein the subject has not yet exhibited glucocorticoid-induced side-effects.

6. The method of claim 1, wherein the glucocorticoid-induced side-effects are selected from osteoporosis, skin atrophy, muscle atrophy, metabolic disorder, and endocrine disorder.

7. The method of claim 1, wherein the PI3K/Akt/mTOR inhibitor is selected from sirolimus, wortmannin, alvocidib, AZD8055, triptolide, WYE-125132, OSI-027, CGP-60474, PI-103, BMS-387032, emetine, NVP-BEZ235, torin 1, LY294002, QL-X-138, and mitoxantrone.

8. The method of claim 1, wherein the PI3K/Akt/mTOR inhibitor is selected from:

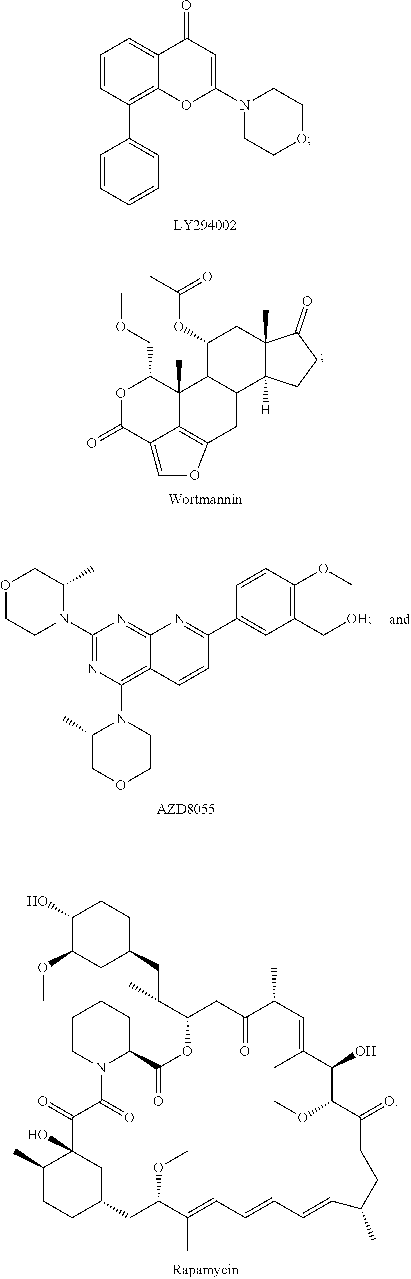

9. The method of claim 1, wherein the PI3K/Akt/mTOR inhibitor is a PI3K inhibitor, an Akt inhibitor, or an mTOR inhibitor.

10. A kit or pharmaceutical composition comprising an PI3K/Akt/mTOR inhibitor and a glucocorticoid agent.

11. A composition comprising a PI3K/Akt/mTOR inhibitor for use in a method of ng, preventing, and/or reducing glucocorticoid-induced side-effects in a subject.


SEQUENCE LISTING (26 sequences)

SEQ ID NO: 1 - DNA, 21 bp, Artificial sequence
  Primer
cagcatcgctctgttcctgta

SEQ ID NO: 2 - DNA, 21 bp, Artificial sequence
  Primer
ctgcgttttcatggagtctca

SEQ ID NO: 3 - DNA, 21 bp, Artificial sequence
  Primer
acccagaaactggtcatcagc

SEQ ID NO: 4 - DNA, 23 bp, Artificial sequence
  Primer
ctgcaatacacacactcatcact

SEQ ID NO: 5 - DNA, 22 bp, Artificial sequence
  Primer
gccctggtggctggaattgacc

SEQ ID NO: 6 - DNA, 22 bp, Artificial sequence
  Primer
ttgcgcttcaaagctgggtccc

SEQ ID NO: 7 - DNA, 21 bp, Artificial sequence
  Primer
ctaccttccgcagtgctccta

SEQ ID NO: 8 - DNA, 19 bp, Artificial sequence
  Primer
cccagccaagaaacggtcc

SEQ ID NO: 9 - DNA, 20 bp, Artificial sequence
  Primer
gctggagcccgtgaaaaaga

SEQ ID NO: 10 - DNA, 19 bp, Artificial sequence
  Primer
ctccgcctctggcattttg

SEQ ID NO: 11 - DNA, 23 bp, Artificial sequence
  Primer
ctgctcatctatacacggttacc

SEQ ID NO: 12 - DNA, 22 bp, Artificial sequence
  Primer
ggaaacgtcgtacagttctgtg

SEQ ID NO: 13 - DNA, 21 bp, Artificial sequence
  Primer
gaatggtgaggaaacgccgat

SEQ ID NO: 14 - DNA, 23 bp, Artificial sequence
  Primer
tgccaagactaaagacaaatggt

SEQ ID NO: 15 - DNA, 22 bp, Artificial sequence
  Primer
aacaccgaaatgtatcagaccc

SEQ ID NO: 16 - DNA, 21 bp, Artificial sequence
  Primer
tgtccagcttaacggaaacca

SEQ ID NO: 17 - DNA, 22 bp, Artificial sequence
  Primer
cgtctatcgggaaggagccaat

SEQ ID NO: 18 - DNA, 24 bp, Artificial sequence
  Primer
gctggataaattcacatgcgtcca

SEQ ID NO: 19 - DNA, 22 bp, Artificial sequence
  Primer
gaaacacgcctccgaaaagagg

SEQ ID NO: 20 - DNA, 22 bp, Artificial sequence
  Primer
gaaagggccgttcacctgtatg

SEQ ID NO: 21 - DNA, 21 bp, Artificial sequence
  Primer
accaccaccgtgttcaacttc

SEQ ID NO: 22 - DNA, 20 bp, Artificial sequence
  Primer
tgggagaggtcgtaatgggg

SEQ ID NO: 23 - DNA, 23 bp, Artificial sequence
  Primer
tagcctttgggaccgcttctcgt

SEQ ID NO: 24 - DNA, 23 bp, Artificial sequence
  Primer
caggtaagccgtgtcttcctccg

SEQ ID NO: 25 - DNA, 20 bp, Artificial sequence
  Primer
accgctacccccgcaaagtg

SEQ ID NO: 26 - DNA, 20 bp, Artificial sequence
  Primer
cccgtcgggccttgcgttta